SEQUENCE LISTING (36 sequences)

SEQ ID NO: 1 - protein, 8 aa, synthetic construct
STSGMGVG

SEQ ID NO: 2 - protein, 16 aa, synthetic construct
HIWWDDVKRYNPALKS

SEQ ID NO: 3 - protein, 8 aa, synthetic construct
TLPHFHDY

SEQ ID NO: 4 - protein, 118 aa, synthetic construct
QVTLKVSGPGILQPSQTLSLTCSFSGFSLNTSGMGVGWIRQPSGKGLEWLAHIWWDDVKRYNPALKSRLTISKDTSGSQVFLKIASVDTADTATYYCARTLPHFFDYWGQGTTLTVSS

SEQ ID NO: 5 - protein, 10 aa, synthetic construct
RASSSVSYMY

SEQ ID NO: 6 - protein, 7 aa, synthetic construct
RTSNLAS

SEQ ID NO: 7 - protein, 11 aa, synthetic construct
KQYHSYPPTWT

SEQ ID NO: 8 - protein, 109 aa, synthetic construct
DIQMTQSPAIMSASPGEKVTISCRASSSVSYMYWYQQKSGSSPKPWIYRTSNLASGVPARFSGSGSGTSYSLTISSMEAEDAATYYCKQYHSYPPTWTFGGGTKLEIKR

SEQ ID NO: 9 - DNA, 19 bp, synthetic construct
  source 1..19: mol_type = other DNA
gtgaagagccscrtgtgct

SEQ ID NO: 10 - DNA, 20 bp, synthetic construct
  source 1..20: mol_type = other DNA
gctcagggttaaggttagcc

SEQ ID NO: 11 - DNA, 22 bp, synthetic construct
  source 1..22: mol_type = other DNA
tgagtcctccggcccctgaatg

SEQ ID NO: 12 - protein, 118 aa, synthetic construct
QVTLKESGPTLVKPTQTLTLTCTFSGFSLSTSGMGVGWIRQPPGKALEWLAHIWWDDVKRYNPSLKSRLTITKDTSGSQVVLTMTNMDPVDTATYYCARTLPHFHDYWGQGTTLTVSS

SEQ ID NO: 13 - protein, 109 aa, synthetic construct
DIQMTQSPSTLSASVGDRVTITCRASSSVSYMYWYQQKPGKAPKPLIYRTSNLASGVPSRFSGSGSGTEYTLTISSLQPDDFATYYCKQYHSYPPTWTFGGGTKVEIKR

SEQ ID NO: 14 - DNA, 21 bp, synthetic construct
  source 1..21: mol_type = other DNA
cgccgctagaggtgaaattct

SEQ ID NO: 15 - DNA, 21 bp, synthetic construct
  source 1..21: mol_type = other DNA
cattcttggcaaatgctttcg

SEQ ID NO: 16 - DNA, 21 bp, synthetic construct
  source 1..21: mol_type = other DNA
accggcgcaagacggaccaga

SEQ ID NO: 17 - DNA, 27 bp, synthetic construct
  source 1..27: mol_type = other DNA
gagatatgagttggacagagacttgaa

SEQ ID NO: 18 - DNA, 21 bp, synthetic construct
  source 1..21: mol_type = other DNA
ccatgtgggagcctgtctgta

SEQ ID NO: 19 - DNA, 23 bp, synthetic construct
  source 1..23: mol_type = other DNA
ctcccccaggacctgtaccacgc

SEQ ID NO: 20 - DNA, 22 bp, synthetic construct
  source 1..22: mol_type = other DNA
gctcttatccaacacagcacta

SEQ ID NO: 21 - DNA, 21 bp, synthetic construct
  source 1..21: mol_type = other DNA
caccgtagcaccttcactatc

SEQ ID NO: 22 - DNA, 24 bp, synthetic construct
  source 1..24: mol_type = other DNA
agccacaccagaagaaacaaacgc

SEQ ID NO: 23 - DNA, 20 bp, synthetic construct
  source 1..20: mol_type = other DNA
cagtcaaatgggctgatgca

SEQ ID NO: 24 - DNA, 29 bp, synthetic construct
  source 1..29: mol_type = other DNA
aaagggctataaagagaataaggtattct

SEQ ID NO: 25 - DNA, 24 bp, synthetic construct
  source 1..24: mol_type = other DNA
ccctgacgaccacgttgtggttca

SEQ ID NO: 26 - DNA, 18 bp, synthetic construct
  source 1..18: mol_type = other DNA
cgccgcattgaccatcta

SEQ ID NO: 27 - DNA, 30 bp, synthetic construct
  source 1..30: mol_type = other DNA
ttagccaggaggttctcaacaatagtctca

SEQ ID NO: 28 - DNA, 30 bp, synthetic construct
  source 1..30: mol_type = other DNA
tcagacaagattcatctagcactggctgga

SEQ ID NO: 29 - DNA, 20 bp, synthetic construct
  source 1..20: mol_type = other DNA
ctgccacatagcccagttca

SEQ ID NO: 30 - DNA, 25 bp, synthetic construct
  source 1..25: mol_type = other DNA
agaagcgactcttctaaggcatctt

SEQ ID NO: 31 - DNA, 25 bp, synthetic construct
  source 1..25: mol_type = other DNA
tctccacaggagctgcaggccttta

SEQ ID NO: 32 - protein, 118 aa, synthetic construct
QVTLKESGPTLVKPTQTLTLTCTFSGFSLNTSGMGVGWIRQPPGKALEWLAHIWWDDVKRYNPSLKSRLTITKDTSGSQVVLTMTNMDPVDTATYYCARTLPHFFDYWGQGTTLTVSS

SEQ ID NO: 33 - protein, 109 aa, synthetic construct
DIQMTQSPSTLSASVGDRVTITCRASSSVSYMYWYQQKPGKAPKPLIYRTSNLASGVPSRFSGSGSGTEYTLTISSLQPDDFATYYCKQYHSYPPTWTFGGGTKLEIKR

SEQ ID NO: 34 - protein, 120 aa, synthetic construct
QVTLRVSGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSS

SEQ ID NO: 35 - protein, 107 aa, synthetic construct
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKR

SEQ ID NO: 36 - protein, 120 aa, synthetic construct
QVTLRVSGPALVKPTQTLTLTCTFSGFSLSTSGMSVGWIRQPPGKALEWLADIWWDDKKDYNPSLKSRLTISKDTSANQVVLKVTNMDPADTATYYCARSMITNWYFDVWGAGTTVTVSS

(19) United States
(12) Patent Application Publication
(10) Pub. No.: US 2024/0400671 A1
(43) Pub. Date: Dec. 5, 2024

(54) MONOCONAL ANTIBODIES TO IL-25 AND USES THEREOF

(71) Applicants: Richard A. SHIMKETS, Suwanee, GA (US); Nathan BARTLETT, Suwanee, GA (US); Crystal JACKSON, Suwanee, GA (US); Lanier Biotherapeutics, Inc., Suwanee, GA (US)

(72) Inventors: Richard A. SHIMKETS, Commerce, GA (US); Nathan BARTLETT, Wallsend (AU); Crystal JACKSON, Jefferson, GA (US)

(21) Appl. No.: 18/698,621

(22) PCT Filed: Oct. 5, 2022
(86) PCT No.: PCT/US2022/077630

§ 371 Date: Apr. 4, 2024

Related U.S. Application Data
(60) Provisional application No. 63/252,601, filed on Oct. 5, 2021.

(51) Int. Cl. C07K 16/24; A61K 39/00; A61P 37/04

(52) U.S. Cl. CPC C07K 16/244; A61P 37/04; A61K 2039/505; C07K 2317/24; C07K 2317/92; C07K 2317/94

(57) ABSTRACT

Disclosed are anti-IL-25 binding molecules and methods of using said binding molecules in the treatment of rhinoviral infection, coronavirus infection, airway inflammation, rheumatoid arthritis, asthma, osteoarthritis, bone erosion, intraperitoneal abscesses and adhesions, inflammatory bowel disorder, allograft rejection, psoriasis, certain types of cancer, angiogenesis, atherosclerosis, cystic fibrosis and multiple sclerosis. Also disclosed are methods of using said binding molecules in the treatment of inflammation due to microbial infection, autoinflammatory disease, and/or autoimmune disease including, but not limited to rhinoviral infection, coronavirus infection, airway inflammation, rheumatoid arthritis, asthma, osteoarthritis, bone erosion, intraperitoneal abscesses and adhesions, inflammatory bowel disorder, allograft rejection, psoriasis, certain types of cancer, angiogenesis, atherosclerosis, cystic fibrosis and multiple sclerosis.

This application claims the benefit of U.S. Provisional Application No. 63/252,601, filed on Oct. 5, 2021, which is incorporated herein by reference in its entirety.

### I. BACKGROUND

Interleukin-25 (IL-25), also known as IL-17E, is a cytokine that belongs to the IL-17 cytokine family and is secreted by type 2 helper T cells (Th2) and mast cells. IL-25 induces the production of other cytokines, including IL-4, IL-5 and IL-13, in multiple tissues and stimulates the expansion of eosinophils. IL-25 has been implicated in chronic inflammation associated with the gastrointestinal tract and the IL-25 gene has been identified in a chromosomal region associated with autoimmune diseases of the gut, such as inflammatory bowel disease (IBD). Conventional therapies for treatment of IBD involve either antibiotics or steroid-derived drugs; however these are not currently successful in inducing or maintaining clinical remission in patients. IL-25 has also been shown to be upregulated in samples from patients with asthma, a condition estimated to affect more than 300 million people worldwide; suggesting that overexpression of this cytokine contributes to the pathology of asthma and related conditions. Thus, there is a need for effective antagonists of IL-25 that are useful in the treatment of diseases and conditions characterized by IL-25 overexpression, including asthma and inflammatory bowel disease.

### II. SUMMARY

Disclosed are novel anti-IL25 binding molecules and methods of using the same to treat diseases or inflammatory symptoms associated with a disease.

In one aspect, disclosed herein are isolated anti-IL25 binding molecules comprising a heavy chain variable domain comprising a CDR1, CDR2, and CDR2 as set forth in SEQ ID NO: 1, SEQ ID NO: 2, and SEQ ID NO: 3, respectively (such as, for example, an anti-IL-25 binding molecule comprising a heavy chain variable domain as set forth in SEQ ID NO: 4 or SEQ ID NO: 12). In some aspects, the heavy chain variable domain may comprise a substitution at residue 29 in CDR1 and/or residue 105. For example, the substitution can comprise an asparagine to serine substitution (N29S) and/or Phenylalanine to Histidine substitution (F105H) such as exemplified by SEQ ID NO: 12.

Also disclosed herein are isolated anti-IL25 binding molecules of any preceding aspect, further comprising a light chain variable domain comprising a CDR1, CDR2, and CDR2 as set forth in SEQ ID NO: 5, SEQ ID NO: 6, and SEQ ID NO: 7, respectively (such as, for example, an anti-IL-25 binding molecule comprising a light chain variable domain as set forth in SEQ ID NO: 8 or SEQ ID NO: 13). In some instances, the light chain variable domain can comprise a substitution at residue 105 of SEQ ID NOL 8 from a leucine to a valine (L105V) such as set forth in SEQ ID NO: 13.

In one aspect, disclosed herein are isolated anti-IL25 binding molecules comprising a light chain variable domain comprising a CDR1, CDR2, and CDR2 as set forth in SEQ ID NO: 5, SEQ ID NO: 6, and SEQ ID NO: 7, respectively (such as, for example, an anti-IL-25 binding molecule comprising a light chain variable domain as set forth in SEQ ID NO: 8 or SEQ ID NO: 13). In some instances, the light chain variable domain can comprise a substitution at residue 105 from a leucine to a valine (L105V).

Also disclosed herein are methods of treating, inhibiting, reducing, decreasing, and/or ameliorating an IL-25 mediated inflammatory disease or condition or IL-25 mediated inflammation associated with a disease or condition (such as, for example, a rhinoviral infection, coronavirus infection, airway inflammation, rheumatoid arthritis, asthma, osteoarthritis, bone erosion, intraperitoneal abscesses and adhesions, inflammatory bowel disorder, allograft rejection, psoriasis, certain types of cancer, angiogenesis, atherosclerosis, cystic fibrosis and/or multiple sclerosis) in a subject comprising administering to the subject a therapeutically effective amount of any of the IL-25 binding molecules of any preceding aspect.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a summary of B cells removed from a mouse immunized with IL-25 and their fluorescence sort plot of B cells binding to IL-25.

FIG. 2 shows denatured gel electrophoresis of antibodies: Lane 1: a human IgG4 positive control, Lane 2: Kaleidoscope pre-stained standard protein marker, Lane 3:

109 (now referred to as LNR109), Lane 4: ABM125 (now referred to as LNR125), Lane 5: ABM109.2 (now referred to as LNR109.2).

FIG. 3 shows a HT-29 cellular potency assay of monoclonal antibodies ABM109, ABM122, ABM124, and ABM125 (now, LNR109, LNR122, LNR124, and LNR125, respectively) against IL-25.

FIG. 4 shows HT-29 cellular potency assay of humanized monoclonal antibody ABM109.2 (now referred to as LNR109.2) in IgG1, IgG2 and IgG4 isotypes.

FIG. 5 shows the surface plasmon resonance (SPR) of anti-IL25 monoclonal antibodies and calculated affinity of the antibodies.

FIG. 6 shows the effects of ABM125 (now referred to as LNR125) or control antibody (IgG) administration on total lung infiltrating cells, eosinophils and macrophages in a model of rhinovirus infection on allergic asthma in mice

FIG. 7 shows the amount of detectable rhinovirus RNA in the lungs of mice treated with control antibody (IgG) or ABM125 (now referred to as LNR125).

FIG. 8 shows the amount of detectable IL-5 RNA in the lungs of mice treated with control antibody (IgG) or ABM125 (now referred to as LNR125).

FIG. 9A shows the potency and binding affinity of humanized (CDR-grafted) versions of ABM125 (now referred to as LNR125), ABM125.9 (now referred to as LNR125.9) and ABM125.10 (now referred to as LNR125.10), compared with the chimeric ABM125 (now referred to as LNR125) with fully mouse variable regions. FIG. 9B shows the potency of binding and binding affinity for anti-IL25 antibodies AMB109.2, ABM125, AMB125.38, ABM126, and ABM126.4 (now referred to as LNR109.2, LNR 125, LNR 125.38, LNR 126, and LNR 126.4, respectively).

FIG. 10 shows that anti-IL25 antibody ABM125.38 (now referred to as LNR125.38) significantly reduces type 2 cytokines and inflammatory cells and inflammatory cells increased in allergic mice and in mice with RV-induced asthma exacerbations.

FIGS. 11A, 11B, 11C, and 11D show localization and production of IL-25 in bronchial epithelial cells. H&E and immunofluorescence staining of IL-25 and IL-17RB in (11A) endoscopic bronchial biopsies or (11B) air-liquid interface-differentiated bronchial epithelial cells (BECs) from healthy donors and donors with asthma. IL-25 (11C) mRNA and (11D) cell associated protein from RV-A1 infected BECs from donors with asthma at 2- and 4-days post infection. Representative of n=5 endoscopic bronchial biopsies and BEC cultures, n=14 RV infected BECs, data analysed by Wilcoxon matched pairs test * p<0.05.

FIGS. 12A, 12B, 12C, and 12D show IL-25 blockade increased IFN production during RV-A1 infection in differentiated BECs from individuals with asthma. Differentiated primary bronchial epithelial cells (BECs) from donors with asthma, were treated with either 10 μg/mL of LNR125 (formerly known as ABM125) or isotype control antibody (LNR2) and infected with RV-A1 and harvested for RNA and supernatants 4 d.p.i. Isolated RNA underwent transcriptomic analysis. (12A) Volcano plot of differentially expressed genes (DEG) between LNR125 and LNR2 treated RV-infected BECs. (12B) Differentially expressed and (12C) secretion of IFN-β and IFN-λ from asthmatic BECs. (12D) RV viral load in LNR 125 and LNR2 treated RV-infected BECs. n=6 asthmatic BECs for volcano plot and viral load. n 9 and, n=7 asthmatic BECs for IFN-λ1/3 and IFN-β ELISA, respectively. (12B) median+/−IQR analysed by Wilcoxon-signed rank test and (12C) Friedmen multiple comparisons test, mean with SD, * P <0.05, ** P<0.01, ns=not significant.

FIGS. 13A, 13B, 13C, 13D, 13E, and 13F show IL-25 negatively regulates antiviral immunity during RV-A1 infection. Differentiated BECs from nine donors with asthma were treated with 10 μg/mL of LNR125 or LNR2, infected with RV-A1 then harvested for total cellular RNA and cellular protein at day 4 p.i. (13A) IL-25 and IL-17RB gene expression and (13B) IL-17RB protein expression (n=7 subset of nine asthma donors) measured by immunoblot and quantitation by densitometry using β-actin as loading control. Alternatively, expanded BECs from two healthy donors were differentiated at ALI, treated with 50 ng/ml rhIL-25 and then infected with RV-A1. Total cellular RNA and apical media was collected at day 4 p.i. (13C) RV viral load was analysed by Taqman qPCR assay. FIG. 13D shows expression of IFN-β and IFNλ2/3 mRNA and (13E) expression of IFN-β- and IFNλ-proteins and (13F) CXCL10 and TNF-α in apical media quantitated using by LEGENDplex. n 9 median +/−IQR, analysed by (13A-13B) Wilcoxon matched-pairs t-test (13C) One-way ANOVA with Holm-Sidak multiple comparisons test mean with SD * P<0.05, ** P<0.01, ns=not significant

FIGS. 14A, 14B, 14C, and 14D show IL-25 blockade augments IFN-λ expression during 229E infection in healthy BECs. Healthy ALI-differentiated CR cells were pre-treated with LNR125 or LNR2 1 day prior to infection with 229E. 229E viral load was quantified by Taqman qPCR analysis and supernatants were collected in PBS at 72 hours post infection and cytokines were analysed using LEGENDplex. n 10 biological replicates of two healthy CR 20) donors, mean with SD analysed by (4A) paired T-test, (4B-4D) one-way ANOVA * P 0.05, ** P<0.01, *** P<0.001

FIGS. 15A, 15B, and 15C show IL-25 blockade reduced allergic-RV-A1 induced type-2 cytokine induction. Mice were sensitised and challenged to ovalbumin (OVA) and on the second day of challenge, treated with LNR125 or LNR2 intraperitoneally followed by infection with RV-A1, 6 hours post final challenge. FIG. 15A shows a schematic of treatment and infection time course. FIGS. 15B and 15C show lung tissue was collected day 1 and day 7 (5A) post infection for Th2 cytokine analyses by ELISA. Bars represent mean+SEM. * P<0.05, ** P<0.01, *** P<0.001, **** P<0.0001 by (5B-5C) 1-way ANOVA with Holm-Sidak's correction for multiple analyses

FIGS. 16A and 16B show IL-25 blockade enhanced IFN-secretion and reduced viral load in allergic mice and mice with RV-induced exacerbations. Mice were sensitised and challenged to ovalbumin (OVA). On the same day as second challenge dose, mice were treated with LNR125 or LNR2, intraperitoneally followed by infection with RV-A1, 6 hours post final challenge. FIG. 16A shows lung tissue was collected one day post infection for IFN analysis by ELISA. FIG. 16B shows viral load was quantified by Taqman qPCR analysis of apical lung lysates. Bars represent mean+SEM. * P<0.05, ** P<0.01, *** P<0.001, **** P<0.0001 by (16A) or 1-way ANOVA with Holm-Sidak's correction for multiple analyses (16B) or Mann Whitney test for non-parametric analysis.

FIGS. 17A, 17B, 17C, 17D, and 17E show IL-25 blockade reduced immune cell infiltration at peak- and resolution of inflammation in allergic mice and mice with RV-induced exacerbation. Mice were sensitised and challenged to ovalbumin (OVA). On the same day as second challenge dose, mice were treated with LNR125 or LNR2, intraperitoneally followed by infection with RV-A1, 6 hours post final challenge. Bronchoalveolar lavage (BAL) was performed to innumerate immune cell counts at day 1- and 7-post infection. (17A) Total BAL counts, (17B) macrophage, (17C) neutrophil, (17D) lymphocyte and (17E) eosinophil cell counts. * P<0.05, ** P<0.01, *** P<0.001, **** P<0.0001 by mixed effects model of 2-way ANOVA with Holm-Sidak's correction for multiple analyses. P values >0.05 denoted by exact P values and broken lines.

FIGS. 18A and 18B show secondary control for endoscopic bronchial biopsies and ALI-differentiated BECs. Immunofluorescence staining of anti-IL-25 and anti-IL-17RB secondary antibodies in (18A) endoscopic biopsies and (18B) ALI-BECs to confirm the specificity of secondary antibodies. Representative of n=5 biopsies.

FIGS. 19A, 19B, and 19C show LNR125 treatment increased antiviral gene expression. RNA was harvested 4 d.p.i from n=6 RV-A1 infected ALI-differentiated BECs treated with LNR125 or LNR2. Isolated RNA underwent transcriptomic analysis. FIGS. 19A and 19B show differentially expressed anti-viral genes. FIG. 19C shows an immunoblot validation and densitometry of antiviral genes, representative blot. Bars are representative of median with IQR analysed by Wilcoxon matched-pairs t-test. light gray and dark gray indicate LNR2 and LNR125 treatment, respectively. * P<0.05, ns=not significant.

FIG. 20 shows persistent replication/shedding of OC43 in upper and lower respiratory tract.

FIG. 21 shows the number of neutrophils/mL and lymphocytes/mL post infection with OC43 or UV OC43.

FIG. 22 shows that IL-25 is the only cytokine induced by betacoronavirus OC43.

FIG. 23 shows a sequence alignment of residues 1-99 of the anti-IL25 antibody LNR125 heavy chain variable domain (SEQ ID NO: 4) and IGHV8-8, IGHV8-5, and IGHV8-12.

FIG. 24 shows a sequence alignment of LNR125 heavy chain variable domain (SEQ ID NO: 4), LNR125.1H grafted heavy chain variable domain (SEQ ID NO: 32), palivizumab heavy chain variable domain (SEQ ID NO: 36), and motavizumab heavy chain variable domain (SEQ ID NO: 34).

FIG. 25 shows a sequence alignment of residues 1-94 of the anti-IL25 antibody LNR125 light chain variable domain (SEQ ID NO: 8) and IGKV1-16.01, IGKV1-16.02, and IGKV1-5.03.

FIG. 26 shows a sequence alignment of LNR125 light chain variable domain (SEQ ID NO: 8), LNR125.1L grafted light chain variable domain (SEQ ID NO: 33), and motavizumab light chain variable domain (SEQ ID NO: 35).

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10”as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular anti-IL-25 binding molecule is disclosed and discussed and a number of modifications that can be made to a number of molecules including the anti-IL-25 binding molecule are discussed, specifically contemplated is each and every combination and permutation of anti-IL-binding molecule and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

The role of type-2 immunity has been recognized by the development of biologics (monoclonal antibodies mAbs) that inhibit interleukin 4 (IL-4), IL-5, or IL-13 and reduce frequency of exacerbations up to 50%. More recently focus has shifted to airway epithelial cell-expressed cytokines such as TSLP that stimulate type 2 immune pathways. IL-25 is also expressed by epithelial cells and stimulates type-2 inflammation. IL-25 expression is higher at baseline and during RV infection in individuals with asthma. IL-25 signals through an IL-17RA/IL-17RB heterodimer receptor on immune cells such as type-2 innate lymphoid cells (ILC2), T helper 2 (Th2) cells, eosinophils, basophils, mast cells as well as bronchial epithelial cells (BECs) which constitutively express IL-25 for immediate secretion upon exposure to proteases or pathogens. In one aspect, disclosed herein are isolated anti-IL25 binding molecules.

### 1. Binding Molecules

As used herein the term “binding molecule” refers to any immunotoxin or immunoglobulin including monoclonal antibodies, polyclonal antibodies, chimeric antibodies, diabodies, nanobodies, humanized or human antibodies, as well as antibodies fragments and functional variants including antigen-binding and/or variable domain comprising fragment of an immunoglobulin that competes with the intact immunoglobulin for specific binding to the binding partner of the immunoglobulin, e.g. IL-25. In some instances the anti-IL-25 binding molecules are broadly neutralizing antibodies.

n one aspect, disclosed herein are isolated anti-IL25 binding molecules comprising a heavy chain variable domain comprising a CDR1, CDR2, and CDR2 as set forth in SEQ ID NO: 1, SEQ ID NO: 2, and SEQ ID NO: 3, respectively (such as, for example, an anti-IL-25 binding molecule comprising a heavy chain variable domain as set forth in SEQ ID NO: 4 or SEQ ID NO: 12). In some aspects, the heavy chain variable domain may comprise a substitution at residue 29 in CDR1 and/or residue 105. For example, the substitution can comprise an asparagine to serine substitution (N29S) and/or Phenylalanine to Histidine substitution (F105H).Also disclosed herein are isolated anti-IL25 binding molecules, further comprising a light chain variable domain comprising a CDR1, CDR2, and CDR2 as set forth in SEQ ID NO: 5, SEQ ID NO: 6, and SEQ ID NO: 7, respectively (such as, for example, an anti-IL-25 binding molecule comprising a light chain variable domain as set forth in SEQ ID NO: 8 or SEQ ID NO: 13). In some instances, the light chain variable domain can comprise a substitution at residue 105 from a leucine to a valine (L105V).

In one aspect, disclosed herein are isolated anti-IL25 binding molecules comprising a light chain variable domain comprising a CDR1, CDR2, and CDR2 as set forth in SEQ ID NO: 5, SEQ ID NO: 6, and SEQ ID NO: 7, respectively (such as, for example, an anti-IL-25 binding molecule comprising a light chain variable domain as set forth in SEQ ID NO: 8 or SEQ ID NO: 13). In some instances, the light chain variable domain can comprise a substitution at residue 105 from a leucine to a valine (L105V).

### a) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with IL-25 such that IL-25 is inhibited from interacting with IL-17RA and/or IL-17RB. Antibodies that bind the disclosed regions of IL-25 involved in the interaction between IL-25 and IL-17RA and/or IL-17RB are also disclosed. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′) 2, Fab′, Fab, Fv, scFv, VHH, nanobody, and the like, including hybrid fragments. Thus, fragments of the antibodies that retain the ability to bind their specific antigens are provided. For example, fragments of antibodies which maintain IL-25 binding activity are included within the meaning of the term “antibody or fragment thereof.” Such antibodies and fragments can be made by techniques known in the art and can be screened for specificity and activity according to the methods set forth in the Examples and in general methods for 25 producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

In some aspects, the anti-IL25 binding molecules disclosed herein can be conjugated to a toxin such as botulinum toxin, tetanus toxoid and the like are conjugated to the binding molecule forming a immunotoxin. In such instances a monoclonal antibody, nanobody, Fab′2, Fab′, scFv or other anti-IL25 binding molecules disclosed herein (such as, for example, anti-IL25 binding molecules comprising a heavy chain variable domain comprising a CDR1, CDR2, and CDR2 as set forth in SEQ ID NO: 1, SEQ ID NO: 2, and SEQ ID NO: 3, respectively (such as, for example, an anti-IL-25 binding molecule comprising a heavy chain variable domain as set forth in SEQ ID NO: 4 or SEQ ID NO: 12) and further comprising a light chain variable domain comprising a CDR1, CDR2, and CDR2 as set forth in SEQ ID NO: 5, SEQ ID NO: 6, and SEQ ID NO: 7, respectively (such as, for example, an anti-IL-25 binding molecule comprising a light chain variable domain as set forth in SEQ ID NO: 8 or SEQ ID NO: 13) are conjugated to the toxin. In some aspects, the heavy chain variable domain may comprise a substitution at residue 29 in CDR1 and/or residue 105. For example, the substitution can comprise an asparagine to serine substitution (N29S) and/or Phenylalanine to Histidine substitution (F105H) such as exemplified by SEQ ID NO: 12. In some instances, the light chain variable domain can comprise a substitution at residue 105 of SEQ ID NOL 8 from a leucine to a valine (L105V) such as set forth in SEQ ID NO: 13.

In some aspects, the anti-IL25 binding molecules disclosed herein can form the targeting receptor of a chimeric antigen receptor (CAR) T cell, NK cell (CAR NK cell), or macrophage (CARMA). In such instances a scFv comprising the anti-IL25 binding molecules disclosed herein (such as, for example, anti-IL25 binding molecules comprising a heavy chain variable domain comprising a CDR1, CDR2, and CDR2 as set forth in SEQ ID NO: 1, SEQ ID NO: 2, and SEQ ID NO: 3, respectively (such as, for example, an anti-IL-25 binding molecule comprising a heavy chain variable domain as set forth in SEQ ID NO: 4 or SEQ ID NO: 12) and further comprising a light chain variable domain comprising a CDR1, CDR2, and CDR2 as set forth in SEQ ID NO: 5, SEQ ID NO: 6, and SEQ ID NO: 7, respectively (such as, for example, an anti-IL-25 binding molecule comprising a light chain variable domain as set forth in SEQ ID NO: 8 or SEQ ID NO: 13) . . . . In some aspects, the heavy chain variable domain may comprise a substitution at residue 29 in CDR1 and/or residue 105. For example, the substitution can comprise an asparagine to serine substitution (N29S) and/or Phenylalanine to Histidine substitution (F105H) such as exemplified by SEQ ID NO: 12. In some instances, the light chain variable domain can comprise a substitution at residue 105 of SEQ ID NOL 8 from a leucine to a valine (L105V) such as set forth in SEQ ID NO: 13. This scFv can be joined to a T cell transmembrane domain via a hinge domain and comprise an intracellular domain comprising a CD3ζ domain. In some aspects, the CAR can further comprise a CD28 signaling domain and/or 4-1BB signaling domain.

b) Human antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J (H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

c) Humanized antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### d) Administration of Binding Molecules

Administration of the anti-IL-25 binding molecules can be done as disclosed herein. Nucleic acid approaches for antibody delivery also exist. The anti-IL25 binding molecules, including antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### 2. Homology/Identity

It is understood that one way to define any known variants and derivatives or those that might arise, of the disclosed genes and proteins herein is through defining the variants and derivatives in terms of homology to specific known sequences. For example, SEQ ID NO: 4 and SEQ ID NO: 12 set forth a particular sequence of an anti-IL25 heavy chain variable domain and SEQ ID NO: 8 and SEQ ID NO: 13 set forth a particular sequence of an anti-IL25 light chain variable domain. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2:482 (1981), by the homology alignment algorithm of Needleman and Wunsch, J. Mol. Biol. 48:443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### 3. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. A preferable stringent hybridization condition for a DNA: DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### 4. Peptides

### a) Protein Variants

As discussed herein there are numerous variants of the IL-25 binding molecules and IL-25 binding CDRs and heavy and light chain variable regions disclosed herein that are known and herein contemplated. In addition, to the known functional strain variants there are derivatives of the IL-25 binding molecules and IL-25 binding CDRs and heavy and light chain variable regions which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn;Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T.E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:4 sets forth a particular sequence of an anti-IL25 heavy chain variable domain and SEQ ID NO: 8 sets forth a particular sequence of an anti-IL-25 light chain variable domain. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2:482 (1981), by the homology alignment algorithm of Needleman and Wunsch, J. Mol Biol. 48:443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is understood that for this mutation all of the nucleic acid sequences that encode this particular derivative of any of SEQ ID NO: 4, SEQ ID NO: 8, SEQ ID NO 12, SEQ ID NO: 13 are also disclosed

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. 1 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### 5. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable . . .

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above. Methods of Treating Inflammatory Disease and Inflammatory Symptoms of a Disease

Airway epithelial cells are also the primary site of respiratory viral infection and are critical to initiating anti-viral immunity. In the lungs, BECs induce an antiviral response through the production of type I interferon-β (IFN-β) and type III IFN-λ which in turn induce expression of IFN-stimulated genes (ISGs) that directly interfere with viral replication, enhance viral antigen presentation, and activate the adaptive immunity. Deficient/delayed type I and type III IFN production by RV-infected BECs from patients with asthma has been identified and this is thought to contribute to enhanced airway inflammation and bronchoconstriction and more severe disease. However, prior to the present disclosure the mechanisms underlying inadequate antiviral immunity in asthma and how this contributes to disease were not well understood. We hypothesized that IL-25 directly regulates BEC innate immunity during viral infection and inhibition of IL-25 (in addition to suppressing type 2 inflammation) increases interferon expression and reduces viral load. To define the role of IL-25 in regulating airway-epithelial cells anti-viral immunity we employed a humanized IL-25 monoclonal antibody (LNR125 (formerly referred to ABM125)) in in vitro and in vivo models of viral infection in asthma. LNR125 upregulated rhinovirus- and coronavirus-induced IFN-β and IFN-λ in differentiated BECs from donors with asthma. Further, LNR125 IL-25 blockade enhanced ISG expression and down-regulated type-2 immune genes. Exogenous IL-25 protein treatment inhibited innate anti-viral immunity in RV-infected differentiated human BECs. We used an established mouse model to determine effect of a single subcutaneous treatment with LNR125 on anti-viral immunity during RV-exacerbation of allergic airways disease. In additional to suppressing inflammation, antibody-mediated IL-25 blockade increased IFN-β expression in airways and reduced lung viral load.

In one aspect, it is understood and herein contemplated that the disclosed anti-IL25 binding molecules can be used to treat inflammatory diseases and/or conditions (including, but not limited to autoimmune disease, autoinflammatory disease, and microbial infections) as well as inflammatory symptoms associated with a disease or condition; wherein the inflammation is mediated by IL-25. Accordingly, disclosed herein are methods of treating, inhibiting, reducing, decreasing, and/or ameliorating an il-25 mediated inflammatory disease or condition or inflammation associated with a disease or condition (such as, for example, a rhinoviral infection, coronavirus infection, airway inflammation, rheumatoid arthritis, asthma, osteoarthritis, bone erosion, intraperitoneal abscesses and adhesions, inflammatory bowel disorder, allograft rejection, psoriasis, certain types of cancer, angiogenesis, atherosclerosis, cystic fibrosis and/or multiple sclerosis) in a subject comprising administering to the subject a therapeutically effective amount of any of the IL-25 binding molecules disclosed herein (such as, for example, anti-IL25 binding molecules comprising a heavy chain variable domain comprising a CDR1, CDR2, and CDR2 as set forth in SEQ ID NO: 1, SEQ ID NO: 2, and SEQ ID NO: 3, respectively (such as, for example, an anti-IL-25 binding molecule comprising a heavy chain variable domain as set forth in SEQ ID NO: 4 or SEQ ID NO: 12) and/or a light chain variable domain comprising a CDR1, CDR2, and CDR2 as set forth in SEQ ID NO: 5, SEQ ID NO: 6, and SEQ ID NO: 7, respectively (such as, for example, an anti-IL-25 binding molecule comprising a light chain variable domain as set forth in SEQ ID NO: 8 or SEQ ID NO: 13).

As noted above, the disclosed anti-IL25 binding molecules can be used to treat autoimmune diseases where IL-25 plays a role in the disease state. Examples of autoimmune diseases include, but are not limited to graft versus host disease, transplant rejection, Achalasia, Acute disseminated encephalomyelitis, Acute motor axonal neuropathy, Addison's disease, Adiposis dolorosa, Adult Still's disease, Agammaglobulinemia, Alopecia areata, Alzheimer's disease, Amyloidosis, Ankylosing spondylitis, Anti-GBM/Anti-TBM nephritis, Antiphospholipid syndrome, Aplastic anemia, Autoimmune angioedema, Autoimmune dysautonomia, Autoimmune encephalomyelitis, Autoimmune enteropathy, Autoimmune hemolytic anemia, Autoimmune hepatitis, Autoimmune inner ear disease (AIED), Autoimmune myocarditis, Autoimmune oophoritis, Autoimmune orchitis, Autoimmune pancreatitis, Autoimmune polyendocrine syndrome, Autoimmune retinopathy, Autoimmune urticaria, Axonal & neuronal neuropathy (AMAN), Baló disease, Behcet's disease, Benign mucosal pemphigoid, Bickerstaff's encephalitis, Bullous pemphigoid, Castleman disease (CD), Celiac disease, Chagas disease, Chronic fatigue syndrome, Chronic inflammatory demyelinating polyneuropathy (CIDP), Chronic recurrent multifocal osteomyelitis (CRMO), Churg-Strauss Syndrome (CSS), Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Diabetes mellitus type 1, Discoid lupus, Dressler's syndrome, Endometriosis, Enthesitis, Eosinophilic esophagitis (EoE), Eosinophilic fasciitis, Erythema nodosum, Essential mixed cryoglobulinemia, Evans syndrome, Felty syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's encephalopathy, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenia purpura (ITP), Inclusion body myositis (IBM), Interstitial cystitis (IC), Inflamatory Bowel Disease (IBD), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus nephritis, Lupus vasculitis, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Ord's thyroiditis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Rheumatoid vasculitis, Sarcoidosis, Schmidt syndrome, Schnitzler syndrome, Scleritis, Scleroderma, Sjögren's syndrome, Sperm & testicular autoimmunity, Stiff person syndrome (SPS), Susac's syndrome, Sydenham chorea, Sympathetic ophthalmia (SO), Systemic Lupus Erythematosus, Systemic scleroderma, Takayasu's arteritis, Temporal arteritis/Giant cell arteritis, Thrombocytopenia purpura (TTP), Tolosa-Hunt syndrome (THS), Transverse myelitis, Type 1 diabetes, Ulcerative colitis (UC), Undifferentiated connective tissue disease (UCTD), Urticaria, Urticarial vasculitis, Uveitis, Vasculitis, Vitiligo, Vogt-Koyanagi-Harada Disease, and Wegener's granulomatosis (or Granulomatosis with Polyangiitis (GPA)). In one aspect, disclosed herein are methods of treating, inhibiting, reducing, decreasing, and/or ameliorating autoimmune diseases or inflammatory symptoms associated with an autoimmune disease administering to a subject with an autoimmune disease a therapeutically effective amount of any of the anti-IL25 binding molecules disclosed herein (such as, for example, anti-IL25 binding molecules comprising a heavy chain variable domain comprising a CDR1, CDR2, and CDR2 as set forth in SEQ ID NO: 1, SEQ ID NO: 2, and SEQ ID NO: 3, respectively (including, but not limited to anti-IL-25 binding molecules comprising a heavy chain variable domain as set forth in SEQ ID NO: 4 or SEQ ID NO: 12) and further comprising a light chain variable domain comprising a CDR1, CDR2, and CDR2 as set forth in SEQ ID NO: 5, SEQ ID NO: 6, and SEQ ID NO: 7, respectively (such as, for example, an anti-IL-25 binding molecule comprising a light chain variable domain as set forth in SEQ ID NO: 8 or SEQ ID NO: 13). In some aspects, the heavy chain variable domain may comprise a substitution at residue 29 in CDR1 and/or residue 105. For example, the substitution can comprise an asparagine to serine substitution (N29S) and/or Phenylalanine to Histidine substitution (F105H) such as exemplified by SEQ ID NO: 12. And/or the light chain variable domain can comprise a substitution at residue 105 of SEQ ID NOL 8 from a leucine to a valine (L105V) such as set forth in SEQ ID NO: 13. In some aspects it is understood that the use of the disclosed anti-IL25 binding molecules may treat the inflammation and symptoms associated with the autoimmune disease without treating the underlying disease state.

The disclosed anti-IL-25 molecules can also be used to treat autoinflammatory diseases. As used herein “autoinflammatory diseases refer to disorders where the innate immune response attacks host cells. Examples of autoinflammatory disorders include, Familial Cold Autoinflammatory Syndrome (FCAS), Muckle-Wells Syndrome (MWS), Neonatal-Onset Multisystem Inflammatory Disease (NOMID) (also known as Chronic Infantile Neurological Cutaneous Articular Syndrome (CINCA)), Familial Mediterranean Fever (FMF) and other cryopyrin-associated periodic syndromes (CAPS), Tumor Necrosis Factor (TNF)-Associated Periodic Syndrome (TRAPS), TNFRSF11A-associated hereditary fever disease (TRAPS11), Hyperimmunoglobulinemia D with Periodic Fever Syndrome (HIDS), Mevalonate Aciduria (MA), Mevalonate Kinase Deficiencies (MKD), Deficiency of Interleukin-1ß (IL-1ß) Receptor Antagonist (DIRA) (also known as Osteomyelitis, Sterile Multifocal with Periostitis Pustulosis), Majeed Syndrome, Chronic Nonbacterial Osteomyelitis (CNO), Early-Onset Inflammatory Bowel Disease, Diverticulitis, Deficiency of Interleukin-36-Receptor Antagonist (DITRA), Familial Psoriasis (PSORS2), Pustular Psoriasis (15), Pyogenic Sterile Arthritis, Pyoderma Gangrenosum, and Acne Syndrome (PAPA), Congenital sideroblastic anemia with immunodeficiency, fevers, and developmental delay (SIFD), Pediatric Granulomatous Arthritis (PGA), Familial Behçets-like Autoinflammatory Syndrome, NLRP12-Associated Periodic Fever Syndrome, Proteasome-associated Autoinflammatory Syndromes (PRAAS), Spondyloenchondrodysplasia with immune dysregulation (SPENCDI), STING-associated vasculopathy with onset in infancy (SAVI), Aicardi-Goutieres syndrome and other Type 1 Interferonopathies, Acute Febrile Neutrophilic Dermatosis, X-linked familial hemophagocytic lymphohistiocytosis, Lyn kinase-associated Autoinflammatory Disease (LAID), and intestinal and skin inflammatory disorders caused by deletion mutation of the carboxy-terminal segment of the NF-κB essential modulator (NEMO). In one aspect, disclosed herein are methods of treating, inhibiting, reducing, decreasing, and/or ameliorating an autoinflammatory disorder or inflammatory symptoms associated with an autoinflammatory disorder comprising administering to a subject with an autoinflammatory disease a therapeutically effective amount of any of the anti-IL25 binding molecules disclosed herein (such as, for example, anti-IL25 binding molecules comprising a heavy chain variable domain comprising a CDR1, CDR2, and CDR2 as set forth in SEQ ID NO: 1, SEQ ID NO: 2, and SEQ ID NO: 3, respectively (including, but not limited to anti-IL-25 binding molecules comprising a heavy chain variable domain as set forth in SEQ ID NO: 4 or SEQ ID NO: 12) and further comprising a light chain variable domain comprising a CDR1, CDR2, and CDR2 as set forth in SEQ ID NO: 5, SEQ ID NO: 6, and SEQ ID NO: 7, respectively (such as, for example, an anti-IL-25 binding molecule comprising a light chain variable domain as set forth in SEQ ID NO: 8 or SEQ ID NO: 13). In some aspects, the heavy chain variable domain may comprise a substitution at residue 29 in CDR1 and/or residue 105. For example, the substitution can comprise an asparagine to serine substitution (N29S) and/or Phenylalanine to Histidine substitution (F105H) such as exemplified by SEQ ID NO: 12. And/or the light chain variable domain can comprise a substitution at residue 105 of SEQ ID NOL 8 from a leucine to a valine (L105V) such as set forth in SEQ ID NO: 13 . . . . In some aspects it is understood that the use of the disclosed anti-IL25 binding molecules may treat the inflammation and symptoms associated with the autoimmune disease without treating the underlying disease state.

IL-25 is also known to be involved in inflammatory responses to microbial infection and inflammatory symptoms associated with a microbial infection. Left unchecked, the microbial inflammation will reach the end stage inflammatory condition known as sepsis. As used herein, “microbial inflammation” refers to a condition associated with its cardinal signs such as redness, swelling, increase in temperature, pain, and impairment of organ function such as disordered respiration as a result of the epithelial injury with adjacent microvascular endothelial injury in the lungs (and other organs) due to a microbial infection such as a virus, bacteria, fungi, or parasite. That is, “Microbial inflammation” is a mechanism of disease caused by infection (“microbial insult”). Microbial inflammation evolves from innate immune response to an infection due to a microbe such as, for example, a virus, bacterium, fungus, or parasite. Thus, the microbial injury caused by microbial virulence factors is aggravated by the host-produced inflammatory mediators that impede the clearance of invading microbes and add insult to organ's injury. It is understood and herein contemplated that the microbial inflammation and its end stage, sepsis can result from any microbial insult elicited by known (or unknown) virulence factors and microbial antigens. While the disclosed anti-IL25 binding molecules may not be effective in eliminating the microbial infection, the disclosed anti-IL25 binding molecules can alleviate the inflammation associated with the infection and thereby prevent sepsis. In other words it is understood that the use of the disclosed anti-IL25 binding molecules may treat the inflammation and symptoms associated with the autoimmune disease without treating the underlying disease state. Accordingly, disclosed herein are methods of treating, inhibiting, decreasing, reducing, and/or ameliorating a microbial infection, IL-25 mediated inflammation resulting from a microbial infection, the inflammatory symptoms of a microbial infection, and/or sepsis in a subject with a microbial infection comprising administering to the subject any of the anti-IL-25 binding molecules disclosed herein (such as, for example, anti-IL25 binding molecules comprising a heavy chain variable domain comprising a CDR1, CDR2, and CDR2 as set forth in SEQ ID NO: 1, SEQ ID NO: 2, and SEQ ID NO: 3, respectively (including, but not limited to anti-IL-25 binding molecules comprising a heavy chain variable domain as set forth in SEQ ID NO: 4 or SEQ ID NO: 12) and further comprising a light chain variable domain comprising a CDR1, CDR2, and CDR2 as set forth in SEQ ID NO: 5, SEQ ID NO: 6, and SEQ ID NO: 7, respectively (such as, for example, an anti-IL-25 binding molecule comprising a light chain variable domain as set forth in SEQ ID NO: 8 or SEQ ID NO: 13). In some aspects, the heavy chain variable domain may comprise a substitution at residue 29 in CDR1 and/or residue 105. For example, the substitution can comprise an asparagine to serine substitution (N29S) and/or Phenylalanine to Histidine substitution (F105H) such as exemplified by SEQ ID NO: 12. And/or the light chain variable domain can comprise a substitution at residue 105 of SEQ ID NOL 8 from a leucine to a valine (L105V) such as set forth in SEQ ID NO: 13.

The innate and adaptive immune response to infecting pathogen (disease-causing microorganism) can include the burst in production of cytokines, chemokines, and proteolytic enzymes by granulocytes, monocytes, macrophages, dendritic cells, mast cells, innate lymphoid cells, T cells, B cells, NK cells, and NK T cells. Microbial inflammation can be localized to a specific organ- or can be systemic. Microbial inflammation can proceed in stages from acute to subacute and chronic with attendant tissue destruction and subsequent fibrosis. Left unchecked, the acute microbial inflammation can lead to sepsis and septic shock, the end stage of microbial inflammation.

“Pathogen” is an agent that causes infection or disease, especially a virus, bacterium, fungus, protozoa, or parasite.

It is understood that the pathogen can be a virus. Thus in one embodiment the pathogen can be selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (such as, for example, avian coronavirus (IBV), porcine coronavirus HKU15 (PorCoV HKU15), Porcine epidemic diarrhea virus (PEDV), Human Coronavirus 229E (HCoV-229E), human coronavirus OC43 (HCoV-OC43), human coronavirus HKUI (HCoV-HKU1), Human coronavirus NL63 (HCoV-NL63), severe acute respiratory syndrome coronavirus (SARS-COV), Middle East respiratory syndrome-related coronavirus (MERS-COV), SARS-COV-2 (including, but not limited to the SARS-COV-2 B1.351 variant, SARS-COV-2B.1.1.7 (alpha), SARS-COV-2B.1.1.7 variant mutant N501Y (alpha), SARS-COV-2 delta variant, SARS-COV-2 P.1 variant, SARS25 CoV-2 with T487K, P681R, and L452R mutations in B.1.617.2 (Delta), SARS-COV-2 with K417N mutation in AY.1/AY.2 (Delta plus), SARS-COV-2 with D614G, P681H, and D950N mutations in B.1.621 (Mu), SARS-COV-2 with G75V, T76I, A246-252, L452Q, F490S, D614G, and T859N mutations in C.37 (Lambda), SARS-COV-2 with T478K, Q498R, and H655Y mutations in B.1.1.529 (Omicron)), Influenza virus A (including, but not limited to HIN1, HIN2, H2N2, H3N1, H3N8, H5N1, H5N2, H5N3, H5N8, H5N9, H7N1, H7N2, H7N3, H7N4, H7N7, H7N9, H9N2, or H10N7), Influenza virus B (including, but not limited to Victoria or Yamagata), Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2. In one aspect, disclosed herein are methods of methods of treating, inhibiting, decreasing, reducing, and/or ameliorating a rhinoviral or coronaviral infection or IL-25 mediated inflammation resulting from a rhinoviral or coronaviral infection (including, but not limited to acute respiratory distress syndrome (ARDS and long COVID) in a subject comprising administering to the subject any of the anti-IL25 binding molecules disclosed herein.

Also disclosed are methods wherein the pathogen is a bacterium. The pathogen can be selected from the group of bacteria consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Mycobacterium chimaera, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species, and Mycoplasma species. In one aspect the bacteria is not Bacillus anthracis.

Also disclosed are methods wherein the pathogen is a fungus selected from the group of fungi consisting of Candida albicans, Cryptococcus neoformans, Histoplasma capsulatum, Aspergillus fumigatus, Coccidiodes immitis, Paracoccidioides brasiliensis, Blastomyces dermitidis, Pneumocystis carinii, Penicillium marneffi, and Alternaria alternata.

Also disclosed are methods wherein the pathogen is a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

### C. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

Transgenic mice overexpressing mouse Ig-Alpha, mouse Ig-Beta and human interleukin 6 were injected intraperitoneally with recombinant human IL-25 (R&D Systems) at 2 week intervals. After a significant immune response was mounted as measured by serum ELISA, the lymph nodes, spleens and bone marrow cells were harvested, B cells surface-expressing IgM isotype antibodies were subtracted with magnetic beads, and the remaining cells were sorted for their ability to bind IL-25, using a MoFlo Fluorescence-Activated Cell Sorter (FIG. 1).

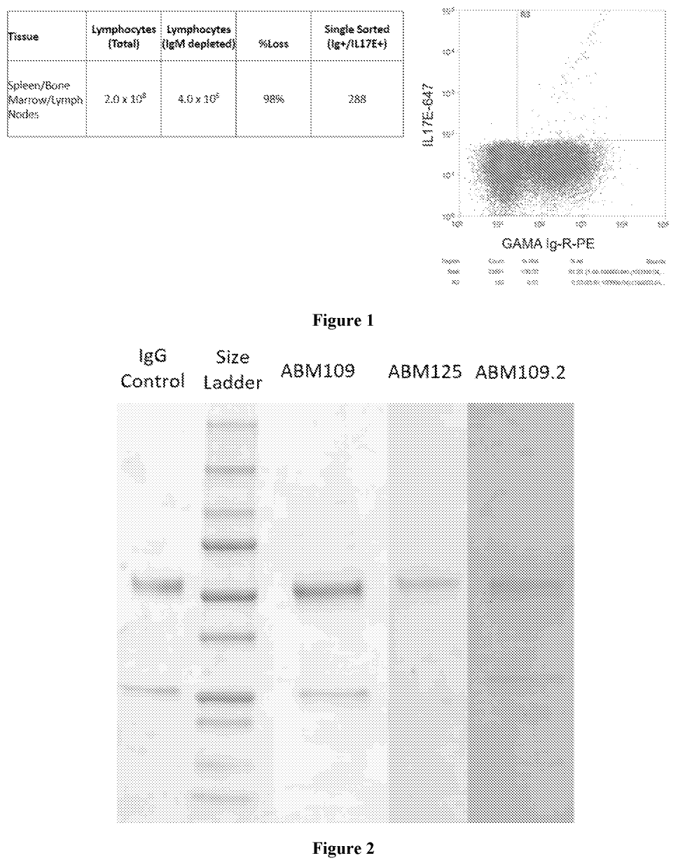

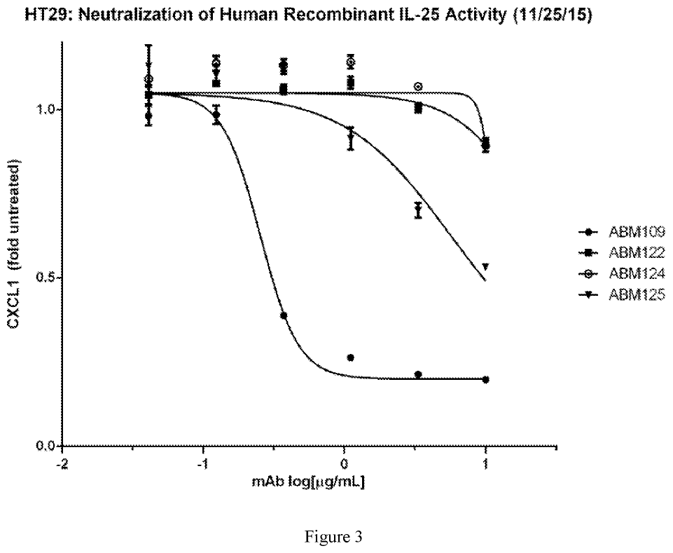

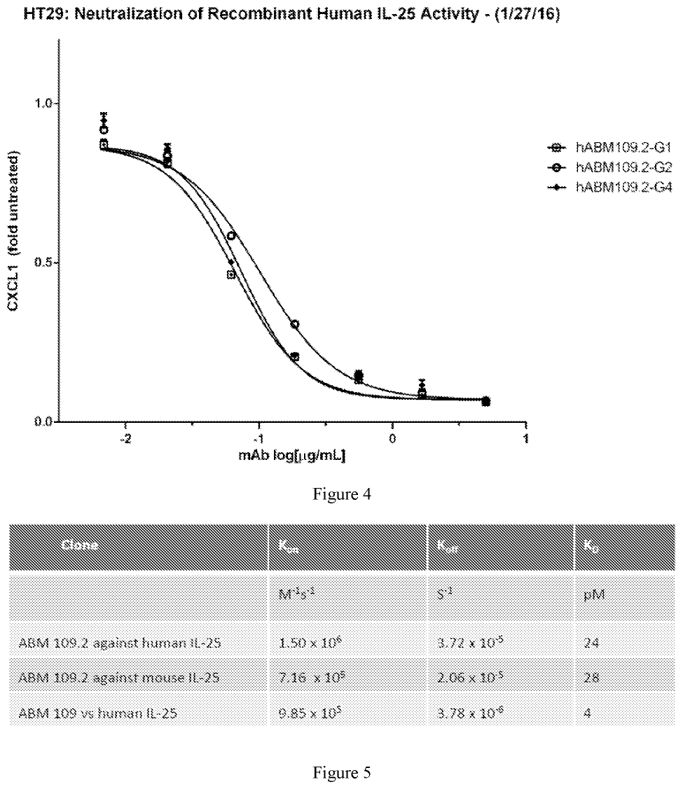

Cells positive for IL-25 binding were sorted into 96-well plates, subjected to single cell RT-PCR to amplify variable regions, and the variable regions were cloned into expression vectors containing either a heavy chain human IgG4 constant region or light chain human IgK constant region. The resulting heavy and light chain clone pairs were transfected into HEK293 cells, and the resulting antibody protein was purified with protein A resin (FIG. 2) and assayed for its ability to neutralize recombinant human IL-25 activity in HT-29 cells (FIG. 3). A humanized version of ABM109 (designated LNR109.2) was also tested for potency in the HT-29 assay (FIG. 4).

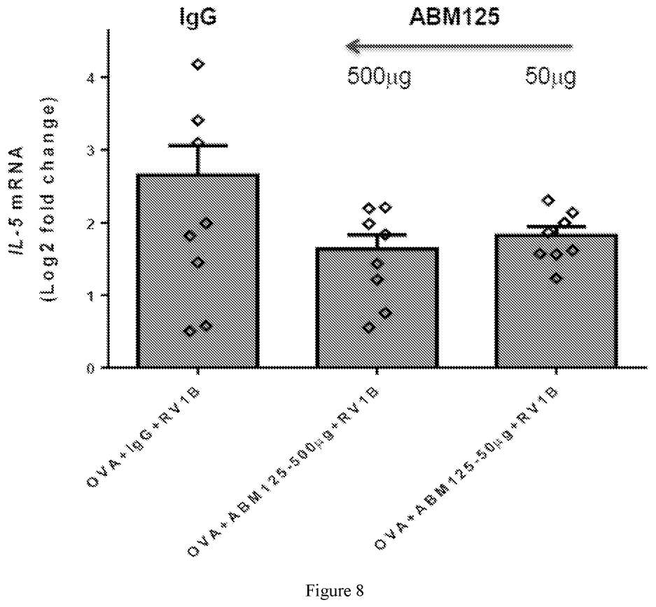

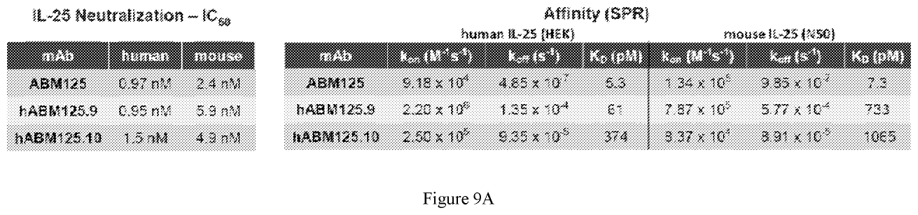

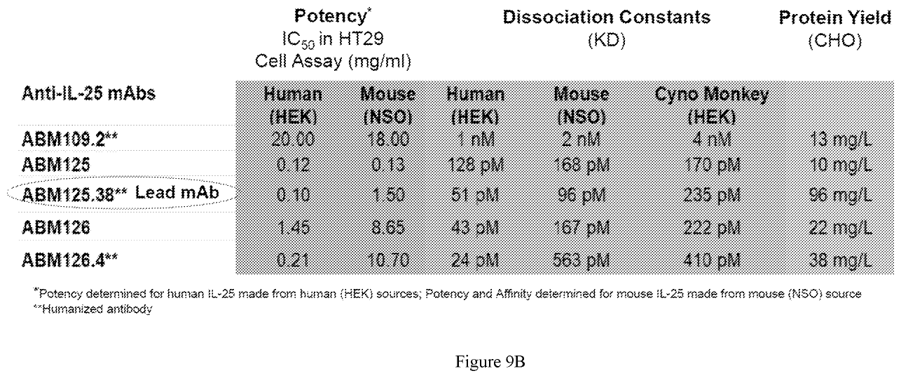

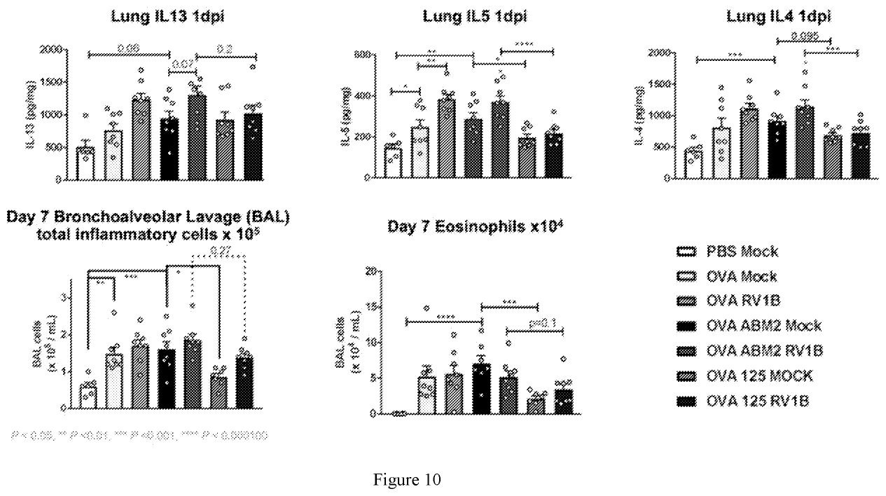

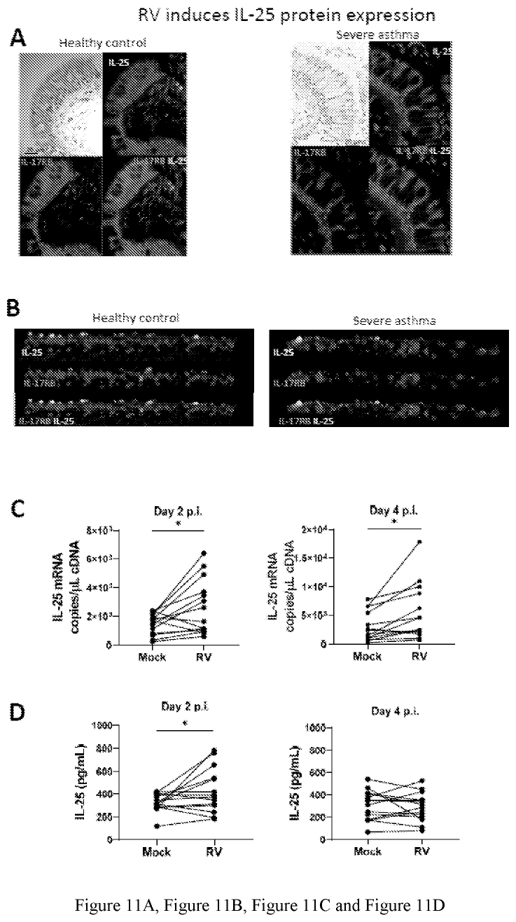

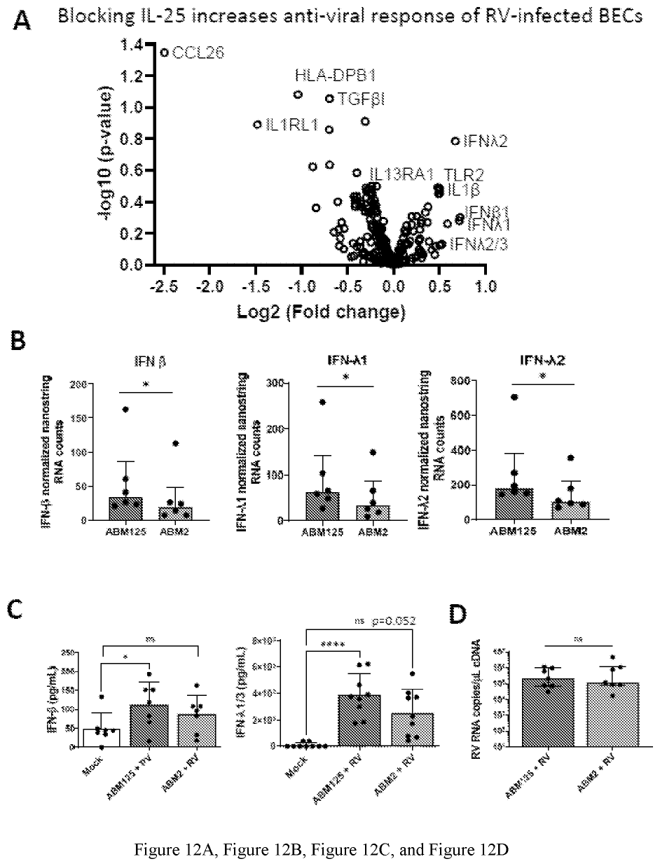

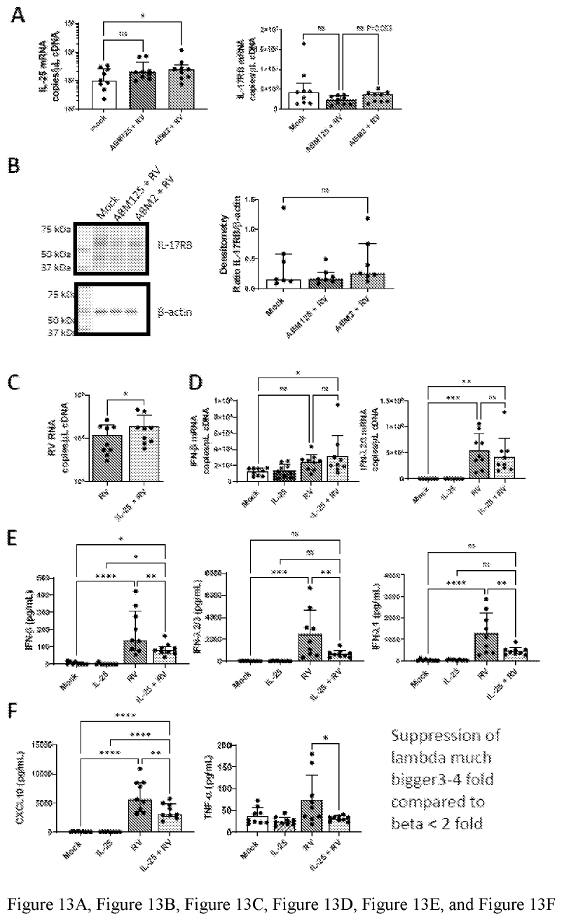

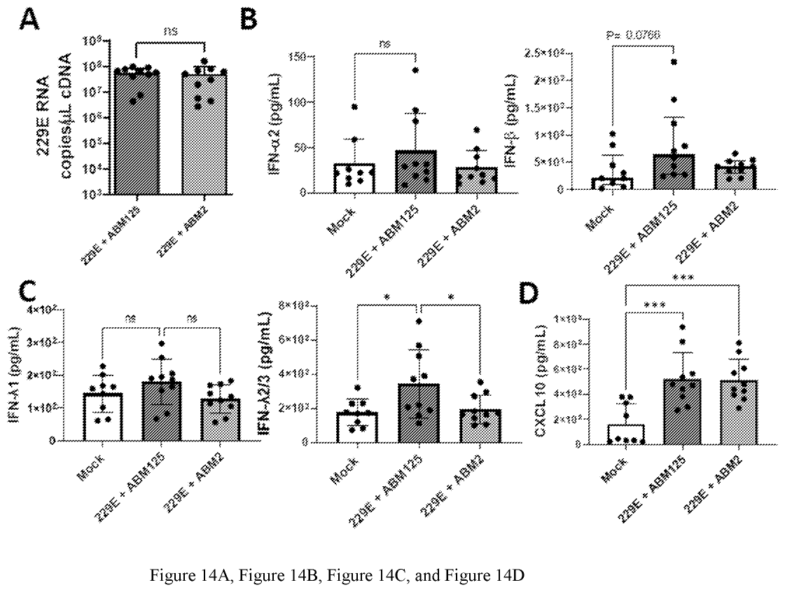

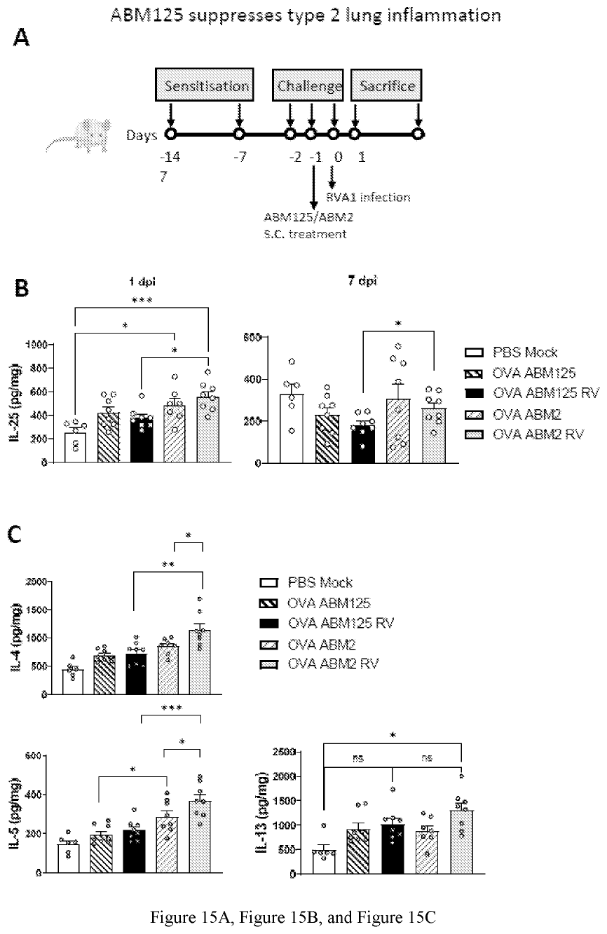

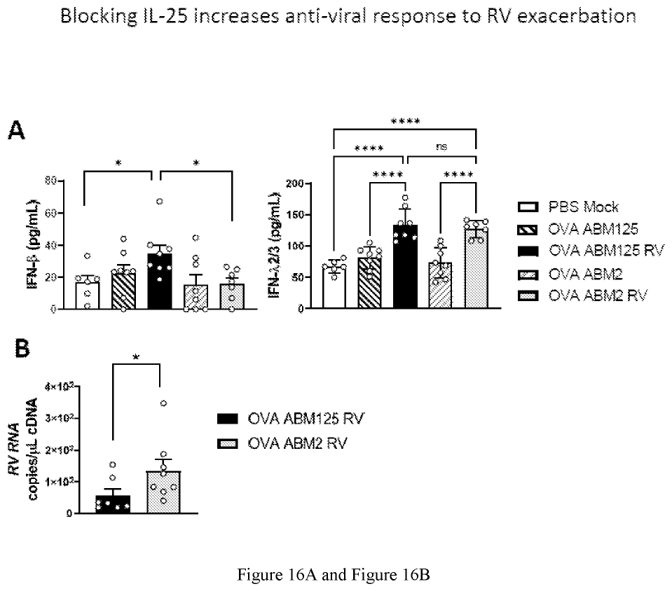

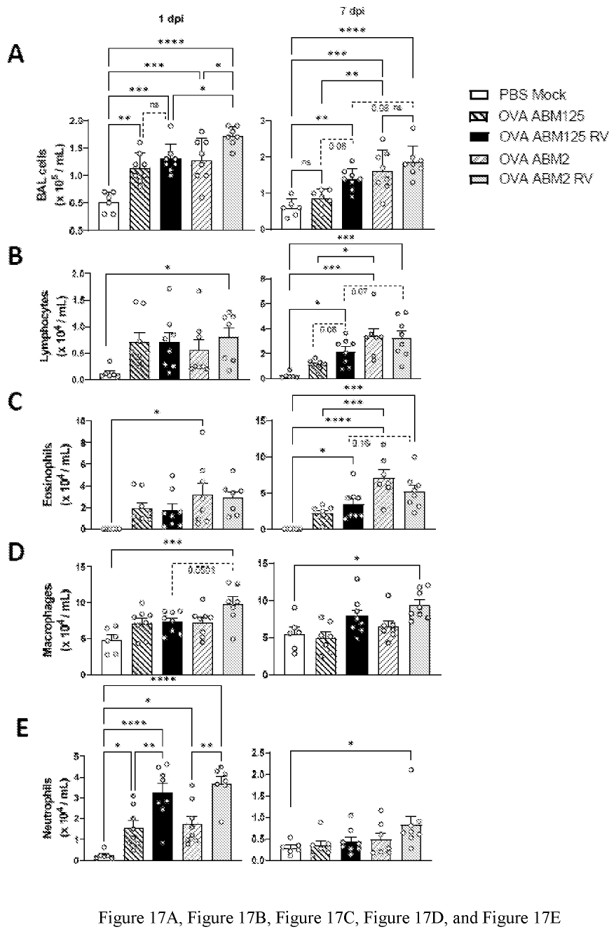

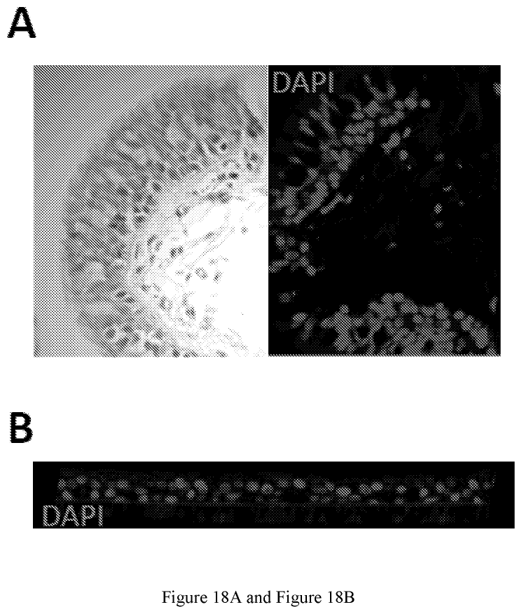

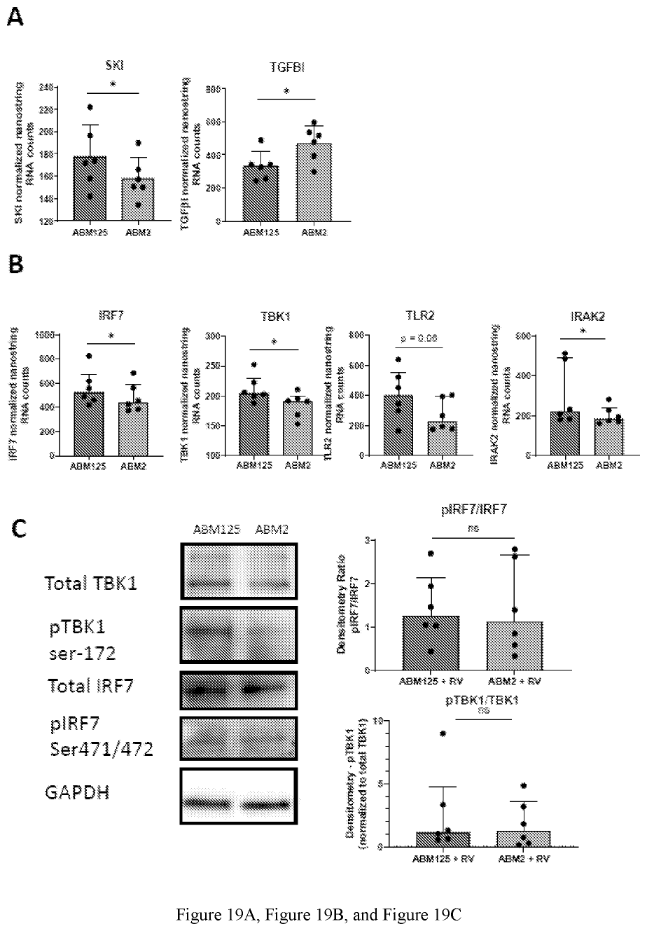

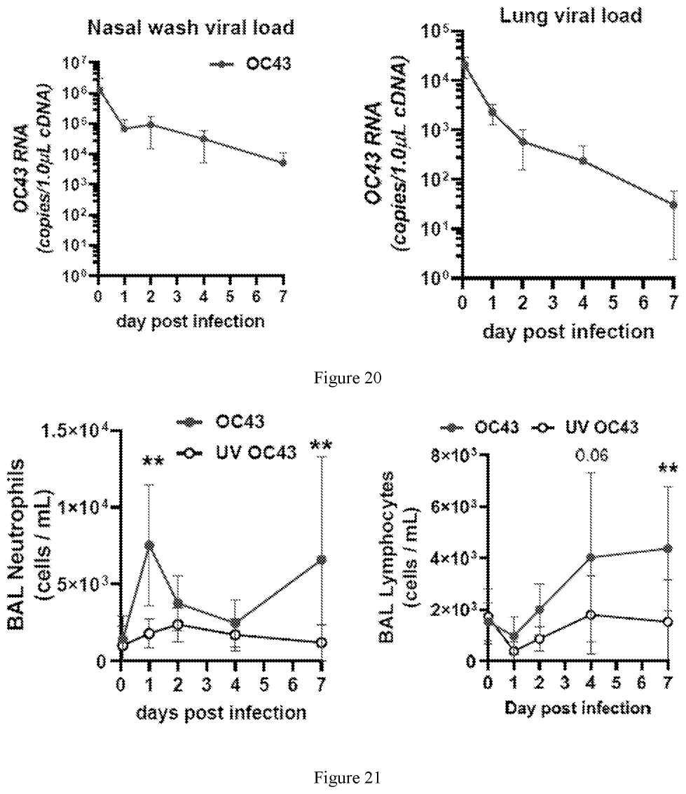

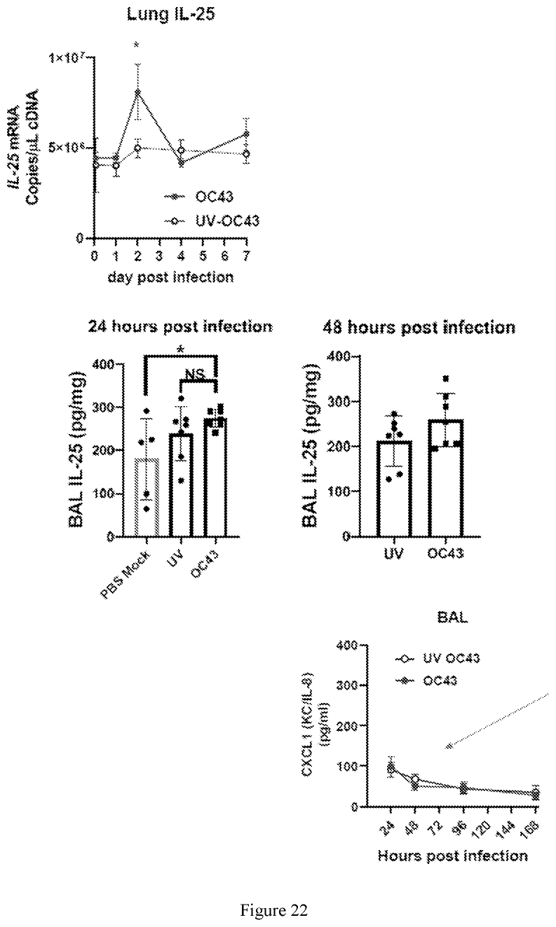

The affinity of the antibodies for IL-25 were measured using Surface Plasma Resonance (SPR) using a Biacore T-100 (GE Healthcare). (FIG. 5, FIG. 9) Antibodies were immobilized to carboxymethylated dextran on flow cells 2-4 on CM5 chip (flow cell 1 used as a reference). rhIL25 or rmIL 15 in HBS-P buffer was run over chip at increasing concentrations (2.5, 5.0, 10, 20, and 40 nM) with a 120 sec injection time for each concentration. After the 5^{th} injection, HBS-P buffer was run through each flow cell and IL-25 was allowed to dissociate for 20 minutes. The surface was regenerated with a 30 second exposure of 10 mM Glycine-HCL pH 1.5. Single cycle kinetic analysis was performed on each antibody with the reference cell subtracted.

LNR125 was purified and administered to mice in a model of rhinovirus infection in allergic asthma. Mice were sensitized with low LPS hen egg ovalbumin (OVA 50 μg in 2 mg alum). Mice were then challenged with 50 μg ovalbumin (OVA) intranasally (i.n.) on three consecutive days. Directly after the final OVA challenge mice were administered intraperitoneally (i.p.) LNR125 or isotype control (IgG). Four hours after mAb dosing mice were infected i.n. with 2.5×10⁶ TCID₅₀ RV1B. Inflammatory responses were assessed at day three post injection. Cellular recruitment was then assessed in bronchoalveolar lavage fluid and lung mRNA expression of IL-5 was assessed by qPCR with SYBR Green chemistry, expressed at Log 2 (fold change) relative to Saline/PBS controls at 3 days post-infection.

Total RNA was extracted from mouse apical lung lobes stored in RNA later (Qiagen), followed by cDNA synthesis (OMNISCRIPT® RT kit, Qiagen). RV-1B genomic RNA primers and probe sequences: sense 5′-GTGAAGAGCCSCRTGTGCT-3′ (SEQ ID NO: 9) 50 nm, antisense 5′-GCTSCAGGGTTAAGGTTAGCC-3′ (SEQ ID NO: 10) 300 nm and probe-5′-FAM-TGAGTCCTCCGGCCCCTGAATG-TAMRA-3′ (SEQ ID NO: 11) 100 nm. An ABI 7500 Taqman (ABI) was used to analyse the PCR reactions. RNA was quantified using a standard curve generated by amplification of plasmid DNA and is expressed as copies per μl of cDNA reaction.

### 2. Example 2: Humanization of Ant-IL25 Antibody

The high-throughput monoclonal antibody discovery process begins with the cloning of mouse variable regions recovered from single B cells into proprietary human constant region-containing vectors, thus generating chimeric antibodies, which are tested for desirable properties. Ideally, multiple functional antibodies are selected to move forward into humanization, because in some cases it is not possible to design successful human framework grafts, while in other cases it is possible to obtain humanized antibodies with properties superior to the parent chimera.

Our approach to humanization comprises the generation of multiple heavy and light chain grafts, and pairwise testing to determine whether functional grafts can be immediately obtained. Structural models of each mouse fAb are generated and compared to the designed grafted fAb structure. As needed, back mutations are made in the grafted constructs in order to attempt to bring the structural models into alignment and maximize retention of parental affinity. Potential post-translational modification sites and other manufacturing challenges, such as non-canonical or unpaired cysteine residues, potential deamidation sites and N-glycosylation sites are identified and engineered away. If additional affinity or expression levels are required, mutagenesis of CDRs is undertaken using proprietary methods.

### A) Heavy Chain Humanization

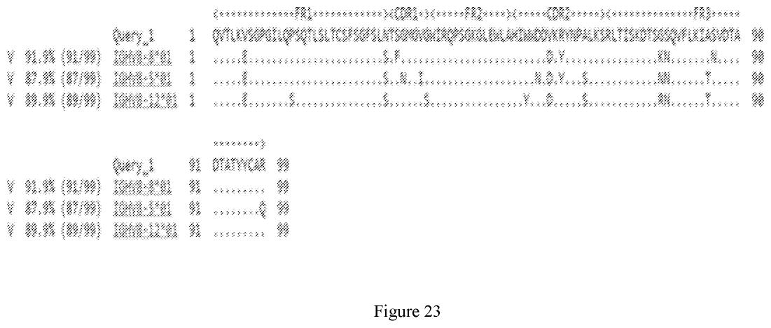

Lead chimeric antibody LNR125 was compared to human scaffolds, and the CDRs were grafted onto the closest human variable region chains. The heavy chain of LNR125 is derived from mouse IGHV8-8, with 8 amino acid substitutions from germline, of which 3 are in CDRs (FIG. 23). One mutation, S29N, resulted in the creation of an N-glycosylation site which was confirmed biophysically.

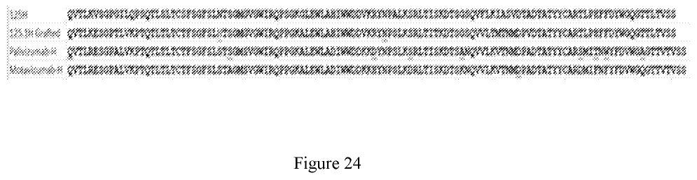

Using published studies on antibody framework residue influence on biophysical properties as a guide (Honegger, et al. 2009 and others), CDRs were grafted onto human IGHV2-5-09 and IGHV4-30-4. When grafted to the IGHV2 heavy chain, the result was only 8 amino acid changes from human germline, whereas grafting to the IGHV4 heavy chain resulted in 18 amino acid changes from human germline. Subsequent testing demonstrated good performance of the graft to the IGHV2, and poor performance of the IGHV4 chains. The successful FDA-approved anti-RSV drug Palivizumab and its derivative Motavizumab, both existing as IGHV2 frameworks, have high expression levels and good stability, and provided a model for our work with humanized LNR125 constructs. FIG. 24 shows a sequence alignment of LNR125, LNR125.1H grafted, palivizumab, and motavizumab.

### b) Light Chain Humanization

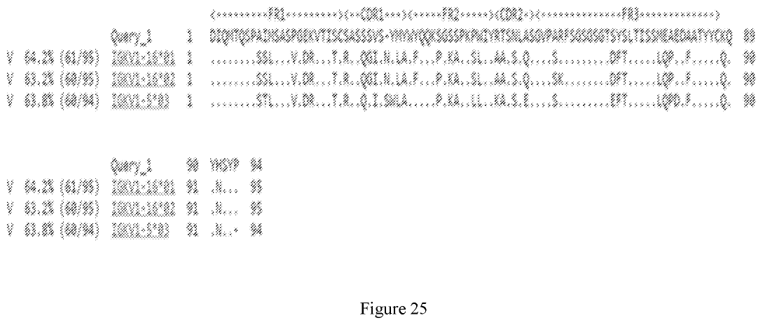

The LNR 125 light chain was derived from the mouse IGKV4-61 and differed from mouse germline in 5 amino acid-changing positions, of which only I was in a CDR. This family is most similar to human IGKV1-5 and IGKV3-15 (FIG. 25).

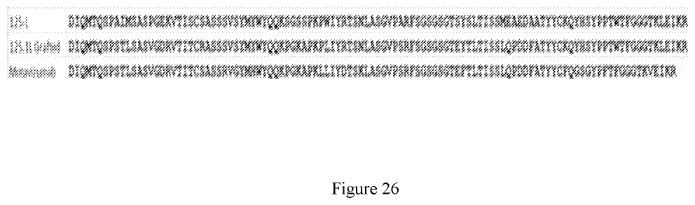

Again using Honegger et al. as a guide, the CDRs from the LNR125 light chain were grafted onto IGKV1-5 with 13 amino acids different from human germline, and on IGKV3-15 with 15 amino acids different from human germline. It was noted that Palivizumab and Motavizumab are human IGKV1-5 derivatives, and so that pairing with the IGH2-5 heavy chain was attractive (FIG. 26)

Upon testing, both light chains performed similarly with the IGHV2 heavy chain in retaining virtually 100% of the mouse chimera potency, while the IGHV4 graft demonstrated a 10×decrease in potency.

### c) Further Engineering to Improve

### Manufacturability

### (1) Glycosylation Site Removal

In order to remove the N-glycosylation site from the heavy chain CDR1, a variety of substitutions were made (S, Q and G) at position N29 to determine whether the activity could be retained with the elimination of the glycosylation site. Using a combinatorial approach, it was determined that reversion of N29 back to the germline serine retained the potency and expression level when paired with the IGKV1 graft but not the IGKV3 graft, so this heavy and light chain pair were moved forward.

(2) Correction of dimerization tendency

Upon detailed biophysical characterization, it was discovered that when the lead antibody was eluted at pH2.5, there was a significant (>50%) ratio of antibody dimer to monomer. This dimer persisted independent of the isotype (IgG1, IgG2, IgG4 tested) and independent of the cell line (CHO, HEK). Hydrophobicity analysis of the variable region sequences revealed that the double phenylalanine motif (F-F) in the heavy chain CDR3 might be responsible for this effect. Therefore, a library of amino acid-changing mutations was introduced at F105 to determine whether potency could be retained in the absence of dimerization and under potentially less harsh (pH3.5) elution conditions. Of the amino acids tested at that position, only one change, F105H, allowed for the production of 99% monomer and retention of activity (see Table 5). The introduced histidine likely disrupts the hydrophobic interactions that may have been occurring between the heavy chain CDR3s of 2 different antibody molecules. The antibody names in Table 5 retain the old nomenclature hABM125.23, hABM125.24, hABM125.25, hABM125.26, hABM125.27, hABM125.28, hABM125.29, hABM125.30, hABM125.31, hABM125.32, hABM125.33, and hABM125.34, but are now referred to as hLNR125.23, hLNR125.24, hLNR125.25, hLNR125.26, hLNR125.27, hLNR125.28, hLNR125.29, hLNR125.30, hLNR125.31, hLNR125.32, hLNR125.33, hLNR125.34, respectively.

TABLE 5

| | | | | Ex- |
|---|---|---|---|---|
| Humanized | Heavy | Light | IC50 | pression |
| Antibody | Chain | Chain | (ug/ml) | (mg/L) |
| ABM125 (Chimera) | ABM125H | ABM125L | 0.18 | 8 |
| hABM125.23 | ABM125.9 (V) | ABM125.1L | 17.7 | 32.2 |
| hABM125.24 | ABM125.9 (T) | ABM125.1L | 37 | 14.7 |
| hABM125.25 | ABM125.9 (G) | ABM125.1L | 2.7 | 16.4 |
| hABM125.26 | ABM125.9 (M) | ABM125.1L | 0.42 | 33.5 |
| hABM125.27 | ABM125.9 (U) | ABM125.1L | 0.61 | 37.4 |
| hABM125.28 | ABM125.9 (S) | ABM125.1L | 1.9 | 30.8 |
| hABM125.29 | ABM125.9 (Q) | ABM125.1L | 2.9 | 82.7 |
| hABM125.30 | ABM125.9 (R) | ABM125.1L | 1.7 | 11.5 |
| hABM125.31 | ABM125.9 (P) | ABM125.1L | 3.6 | 25.3 |
| hABM125.32 | ABM125.9 (I) | ABM125.1L | 2.7 | 41.5 |
| hABM125.33 | ABM125.9 (A) | ABM125.1L | 4.4 | 43.7 |
| hABM125.34 | ABM125.9 (H) | ABM125.1L | 0.22 | 34.7 |

For completeness, a variety of other amino acid changes shown in other antibodies to improve expression were tested. Only one change, light chain L105V, made a positive impact, improving the CHO expression level in our transient system by over 3X to nearly 100 mg/L. This new antibody, designated LNR125.38, was further tested for optimal potency, affinity and biophysical characteristics.

### d) Characterization of LNR125.38: Potency, Affinity and Stability

Our lead humanized candidate LNR125.38 has fulfilled important criteria for efficacy and pharmaceutical development. The following table compares the potency, affinity and expression levels of our lead candidate, compared to the parent chimera and other anti-IL-25 antibodies discovered in our program. (Note: we saw similar potency activity of IgG1, IgG2 and IgG4 isotypes of LNR125.38; the IgG4 isotype is shown FIG. 9B)

### 3. Example 3: IL-25 Blockade Augments Antiviral Immunity During Respiratory Virus Infection

Virus-induced exacerbations are the leading cause of hospitalization and mortality for individuals with asthma. Rhinovirus (RV) has been detected in 50-70% of patients with asthma who present to hospital with an exacerbation. As a result, the mechanisms by which a viral infection worsens asthma are a major research focus with two prevailing concepts emerging-deficient/delayed innate anti-viral immunity associated with excessive type-2 immune activation causing airway inflammation.

### a) Results

### (1) RV-A1 Infection Up-Regulates IL-25 Expression by Differentiated Bronchial Epithelial Cells.

RV infection induced higher levels of IL-25 gene and protein expression by undifferentiated (submerged monolayers) BECs from donors with asthma compared to BECs from healthy donors. Here we used human endobronchial biopsies and differentiated primary human BECs (healthy donors and patients with asthma) to gain insight into co-expression of IL-25 and IL-17RB by differentiated BECs (FIG. 1A and FIG. 8A). We first stained the biopsies with haematoxylin and eosin (H&E) to confirm the presence of intact epithelium. We next compared IL-25 and IL-17RB protein expression using immunofluorescence. IL-25 and IL-17RB expression was predominantly located on the apical, luminal mucosal surface of the airway epithelium. We next determined if ALI-differentiated BEC cultures from healthy- and asthmatic donors exhibited a similar IL-25 and IL-17RB expression pattern to that of bronchial biopsies. Again, IL-25 and IL-17RB expression was highly localized to the apical surface of BEC cultures from healthy donors and donors with severe asthma with evidence of co-localisation in both (FIG. 1B and FIG. 8B). Having determined the ALI-BEC culture system recapitulates in vivo airway mucosal surface expression of IL-25 and IL-17RB, we next determined the effect of RV infection. BECs from n=14 donors with asthma (donor characteristics in Table 3) were cultured at ALI for at least 25 days and infected with RV-A1, MOI=0.1. RV infection increased IL-25 gene expression at 2- and 4 days post-infection (FIG. 1C). We next used an in-house-developed sensitive ELISA to investigate secreted IL-25 from apical supernatants of BEC cultures, however, we did not consistently observe increased secreted IL-25 protein in apical media from RV infected BECs. Having observed cell-associated IL-25 in uninfected bronchial biopsies and ALI-BEC cultures by immunofluorescence, we investigated cell-associated IL-25 from BEC cell lysates which revealed RV infection increased cell associated IL-25 protein on 2 d p.i. (FIG. 1D).

TABLE 3
Clinical Characteristics of differentiated BEC donors

| | Healthy | | Asthma | |
|---|---|---|---|---|
| number, n | 2 | | 14 | |
| Age, years (SD) | 63.5 | (16.2) | 56.7 | (13.7) |
| Gender (M/F) | (0/2) | | (4/10) | |
| Atopy (SPT positive) | 1 | | 9 | |
| Severity | NA | | Mild | (3) |
| | NA | | Moderate | (2) |
| | NA | | Severe | (9) |
| Eosinophil counts (% total | 0.13 | (0.2) | 5.79 | (10.9) |
| sputum cells) (SD) | | | | |
| FEV1, % predicted (SD) | 80 | (9.8) | 77 | (19.7) |
| FVC, % predicted (SD) | 86.5 | (7.8) | 86.5 | (10.3) |
| Daily ICS dose, beclomethasone | NA | | 257 | (90.1) |
| equivalent, ug (SD) | | | | |

FEV1, Forced expiratory volume 1 s;
FVC, Forced vital capacity;
ICS, Inhaled corticosteroid;
SPT, skin prick test

(2) Antibody Blockade of IL-25 Augmented RV-Induced Type V/III IFN Expression by BECs from Patients with Asthma.

Having confirmed that IL-25 was expressed in ALI-differentiated BECs and increased by RV-A1 infection, we next examined if IL-25 was modulating epithelial cell innate anti-viral immunity in a subset (n=6) of BEC from donors with moderate-severe asthma (Table 3). Nanostring immune transcriptomics was used to assess the effect of LNR125 IL-25 blockade. Volcano plot showing up-down regulated genes in LNR125 treated RV infected BECs relative to infected cells treated with isotype control antibody (LNR2) at 4 d.p.i . . . . Of the 500+immune genes in the human immune panel we noted two distinct groups: up-regulated innate immunity-(type I/III IFNs, TLR2, IFR7, TBK-1, IRAK2) and down regulated type 2/immune suppressing-genes (CCL26/eotaxin 3, ILIRL1/ST2, TGFβI) (FIG. 2A, FIG. 9A, and FIG. 9B). Further statistical analyses confirmed that IL-25 inhibition (LNR125 (formerly referred to as ABM125) treatment increased expression of type I (IFN-β) and type III (IFN-A) mRNA (FIG. 2B). We also assessed RV-induce IFN protein expression at 4 d p.i. in n=7 ALI-BECs (moderate to severe asthma) treated with LNR2 (isotype control) or LNR125 IL-25 neutralising antibody. For asthmatic BECs treated with isotype control mAb LNR2, RV did not significantly increase either IFN-β or IFN-λ2/3 protein above baseline (mock-infected cells). In contrast, LNR125 treatment significantly upregulated RV-induced type I/III IFNs compared to mock infected asthmatic BECs (FIG. 2C). Viral replication/load can affect IFN expression. Viral load was not different between LNR125- and isotype control LNR2-treated cells suggesting that IL-25 directly regulates epithelial cell type I/III IFN expression (FIG. 2D). We next compared treatment with LNR125 on phosphorylated and total TBK1- and IRF7-protein expression in cell lysates by immunoblot, however we could not identify differences in protein expression compared to LNR2-treated cells (FIG. 9C). In summary, blockade of IL-25 during RV-A1 infection upregulated IFN gene and protein expression independently of viral load in ALI-differentiated BECS from donors with asthma indicative of improved anti-viral immunity.

### (3) IL-25 Signaling Regulated Epithelial Innate Anti-Viral Immunity

We next investigated the effect of ABM125 (now known as LNR 125) treatment on IL-25 signaling in n=9 ALI-BECs derived from patients with moderate to severe asthma (Table 3). RV infection in the presence of isotype control ABM2 significantly increased IL-25 mRNA. ABM125 treatment reduced RV-induced IL-25 gene expression such that it was not significantly greater than mock-infected cells identifying IL-25 as another type-2 immune gene reduced by IL-25 blockade in RV-infected BECs. We also measured IL-17RB mRNA expression and noted a near significant (P=0.053) reduction in ABM125-treated cells compared to cells treated with isotype control ABM2 antibody (FIG. 3A). We did not detect a significant reduction in IL-17RB protein in ABM125-treated, RV-infected BECs as assessed by immunoblot with protein loading normalized to β-actin for densitometric quantification (FIG. 3B).

To further investigate the role of IL-25 signaling on BEC innate anti-viral immunity in conditionally reprogrammed-expanded and then differentiated BECs derived from two healthy donors, (5 replicate transwells for each donor combined n=10) were treated with recombinant IL-25. This significantly increased RV viral load compared to RV infected, untreated cells at 4 d p.i. (FIG. 3C). RV-induced IFN-β and λ gene expression was not significantly different between IL-25- and un-treated, RV-infected cells, although we did note a trend for increased IFN-β- and reduced IFN-A-gene expression in IL-25 treated cells. The effect on RV-induced type I/III IFN proteins was consistent-IL-25 treatment reduced expression of IFN-B, IFN-λ2/3 and IFN-λ1, with suppression of IFN-λ 3-4 fold compared to IFN-β (FIG. 3E). IL-25 treatment also reduced the IFN-induced chemokine CXCL 10 and completely ablated expression of TNF-α protein indicative of broad innate immune suppression (FIG. 3F).

### (4) LNR125 Increased the Anti-Viral Response of Human Coronavirus 229E-Infected BECs.

To determine if IL-25 suppresses anti-viral immunity to other (than RV) respiratory viruses BECs from two healthy donors were conditionally reprogrammed to expand before returning to standard ALI conditions for differentiation. Five replicate wells each (n=10) were treated with ABM125 then infected with the endemic human coronavirus 229E. We collected cells and apical media at 72 h post-infection We did not observe any changes in 229E viral load (viral RNA) with ABM125 treatment (FIG. 4A) or difference in type I IFN protein expression (IFN-α2a, IFN-β) although we did note near significant increased (P=0.0766) IFN-β (FIG. 4B). However, ABM125 treatment significantly boosted IFN-λ2/3 protein expression (FIG. 4C) but did not increase virus induced CXCL 10 (FIG. 4D). No significant difference in IL-25 mRNA or proteins were detected in this context (FIGS. 4E and 4F).

### (5) LNR125 Treatment Attenuated RV-Induced Type-2 Cytokine Production In Vivo.

RV infection augments aeroallergen-induced lung IL-25 expression associated with increased type 2 lung inflammation and blocking the IL-25 receptor (IL-17RB) prevented RV exacerbation of allergic airways disease. Utilizing this muse model, we determined if subcutaneous treatment with LNR125 (formerly ABM125) or LNR2 isotype control could similarly reduce type-2 airway inflammation (to directly confirm the role of IL-25 in viral exacerbation of allergic airways inflammation) as well as augment anti-viral immunity. Mice were systemically sensitised and intranasally challenged with OVA to establish allergic airways disease. Mice were administered a single subcutaneous dose of ABM125 or ABM2 isotype control and the following day infected intranasally with RV with samples collected at 1 d p.i. and 7 d p.i. (FIG. 5A). In isotype ABM2-treated mice, OVA challenge increased IL-25 lung protein compared to negative controls (PBS mock) and this was increased further by RV infection as predicted. AM125 treatment suppressed both allergen (OVA LNR125)- and allergen +virus (OVA LNR125 RV) induced IL-25 lung protein. LNR125 suppression of allergen and virus induced IL-25 was also evident at 7 d.p.i. (FIG. 5B). We next investigated downstream inflammatory mediators of IL-25 which promote allergic inflammation. RV infection augmented OVA-induced type 2 cytokine (IL-4, IL-5 and IL-13) production in LNR2 treated mice. LNR125 treatment reduced OVA+RV induced IL-4 and IL-5 protein in BAL, with a trend-non significant reduction in IL-13 compared to LNR2-treatmented controls such that only OVA LNR2 RV mice had significantly increased BAL IL-13 compared non-allergic, uninfected (PBS Mock) mice (FIG. 5C).

### (6) LNR125 Treatment Enhanced Anti-Viral Immunity During Viral Exacerbation of Allergic Airways Disease.

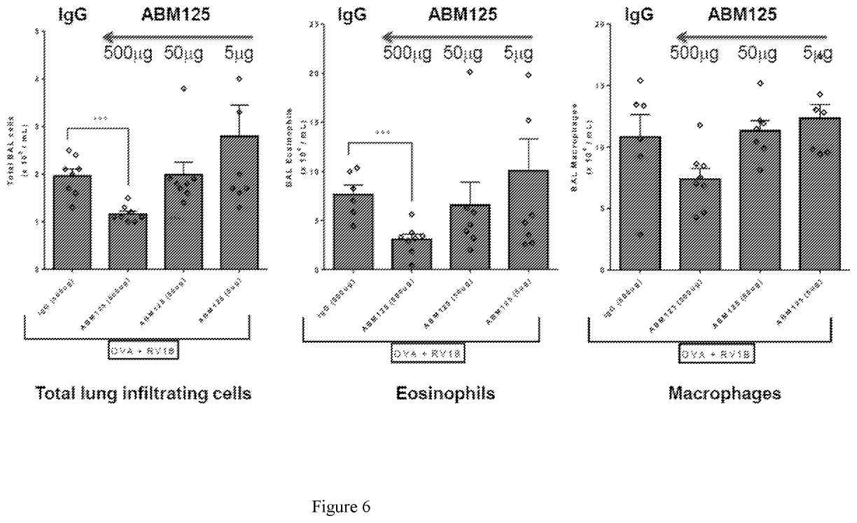

After confirming that LNR125 reduced type-2 cytokines related to RV-exacerbated allergic airways disease, we next evaluated anti-viral IFN proteins in BAL. LNR125 enhanced production of IFN-β in the RV-exacerbated OVA model at 1 d p.i compared to the mock infected and RV-infected LNR2 controls. There was no difference in the level of RV infection induced IFN-λ protein expression in LNR125 and LNR2-treated mice BAL-both RV-infected groups had highly significant induction of IFN-λ2/3 (FIG. 6A). Treatment with LNR125 reduced lung viral load compared to LNR2-treated mice at 1 d p.i. confirming that LNR 125-mediated innate immune re-calibration enhanced anti-viral immunity in vivo (FIG. 6B).

### (7) IL-25 Blockade Reduced Type-2 Airway Inflammation.

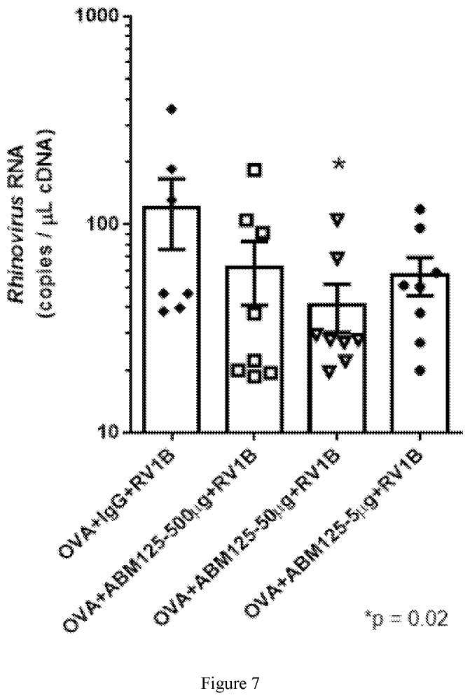

Having examined cytokine and interferon responses we next investigated the role of IL-25 in regulating infiltration of immune cells to determine if the reduced inflammatory cytokines from the BAL corresponded to reduced airway infiltration of cells collected by BAL. RV-infected mice treated with LNR125 had reduced airway infiltration of total cells at 1 d p.i. compared to isotype control antibody LNR2 treated mice (FIG. 7A). In comparison, RV-infected mice treated with OVA-LNR 125 had a trend in reduced total BAL, lymphocytes, and eosinophils at 7 d.p.i (p=0.08, p=0.07, and p=0.16, respectively) compared to RV-infected OVA-LNR2 treated mice (FIG. 7A-C). There was no reduction in macrophages at 1- or 7-d p.i., however macrophages displayed a trend to be reduced in OVA LNR2 RV compared to OVA LNR125 RV (FIG. 7D. Airway neutrophilic inflammation is caused by viral infection and this was reduced in LNR 125 treated mice such that only OVA LNR2 RV mice had increased BAL neutrophils compared to untreated/infected (PBS mock) mice at day 7 p.i. (FIG. 7E).

### b) Materials and Methods

### (1) Ethics Statements

Primary BEC (pBECs) brushings were provided by Professor Peter A. B. Wark (The University of Newcastle) in compliance with the University of Newcastle and Hunter New England Area Health ethics committee (05/08/10/3.09) with written, informed consent prior to collection. Animal experiments were conducted in accordance with the NSW, Australia Animal Research legislation on protocol A-2016-605, reviewed and approved by the University of Newcastle Animal Care and Ethics Committee.

### (2) Air-Liquid Interface Culture of BECs

BECs were obtained from moderate-severe persistent asthmatic donors or donors with GOLD stage 2-3 COPD as defined by asthma and COPD guidelines, respectively. pBECs were cultured until confluent then differentiated at air-liquid interface (ALI). Alternatively, pBECs were obtained from healthy donors and conditionally reprogrammed (CR) with rho-associated protein kinase (ROCK) inhibitor (final concentration 10 μM) in combination with irradiated 3T3 feeder cells in monolayer cultures. CR media consisted of 1:2 ratio of DMEM (high glucose+L-glutamine)/Ham's F12 supplemented with 5% FCS, hydrocortisone (400 30) ng/ml), insulin (5 μg/mL), rhEGF(10 ng/mL), cholera toxin (8.4 ng/ml), adenine (23.9 μg/mL), and 0.2% penicillin streptomycin. Expanded BECs were weaned off the ROCK inhibitor and seeded onto polyester transwell inserts and differentiated. All differentiated donor demographics are described in Table 3. A selection of listed donors was used for IL-25 blockade experiments.

(3) Antibody Treatment with RV-A1 or 229E Infection

Day one prior to infection, the ALI-BECs was treated basally with the anti-IL-25 monoclonal antibody LNR125, or matched IgG isotype control LNR2 (Abeome, USA) at 10 μg/mL in BEBM minimal media (BEBM+1% ITS and 0.5% linoleic acid-BSA) (Lonza, Switzerland). BEC cultures were infected apically with RV-A1 (MOI 0.1) for 2 hours at 35° C. Following infection, virus inoculum was removed, and the apical surface was washed twice with PBS. Minimal media containing LNR125, LNR2, or media was placed apically and refreshed basally, and cells were incubated at 35° C. until day 2 or 4 post infection (d.p.i). Apical supernatants and basal media were collected at indicated time points and stored at −80° C. for downstream protein analysis. Half transwells were lysed in RLT buffer (Qiagen, Germany) containing 1% 2-mercaptoethanol or RIPA buffer containing protease inhibitor cocktail (Roche, Switzerland). Lysate was stored at −80° C.

Separately, 10 μg/mL of LNR125, LNR2, or regular ALI-BEC media was applied apically and basally 1 day prior to infection. Apical media was removed and BECs were infected with 229E (MOI 0.1) 2 hours at 35° C. Following infection, virus inoculum was removed, and the apical surface was washed twice with PBS. Basal media was replaced and supplemented as with RV infection. Apically secreted mediators were collected in PBS on day three and lysates collected as above.

(4) Mouse model

6-8-week-old, wild-type, female BALB/c mice were obtained from Australian Bioresources (ABR, Moss Vale, NSW), sensitised with 50 μg chicken Ovalbumin (OVA) protein in 1% alhydrogel intraperitoneally (i.p.) on day −14 and day −7 followed by intranasal challenge (i.n.) with 50 μg of low LPS OVA in 30 μL of PBS (controls receive PBS alone) on 3 consecutive days (Days −2, −1 and 0) to induce allergic airway inflammation. 250 μg LNR 125 or LNR2 (in 100 μL) was administered subcutaneously (s.c.) on day-1. Mice were then infected i.n. with 2.5×10⁶ TCID₅₀/mL of RV-A1 or mock infected with PBS, 6 hours after final OVA challenge. On day 1 and day 7 post-infection, bronchoalveolar lavage samples (BAL fluid and leukocytes) were collected, apical lung lobe tissue was collected for RNA extraction, and the remaining lung tissue was snap frozen for cytokine analyses.

(5) RNA Extraction and Gene Expression Analysis for Quantitative Real-Time (qRT)-PCR

RNA was extracted using the miRNeasy kit (Qiagen, Germany) following the supplier's protocol. RNA concentrations were determined by Nanodrop and 200 ng (cells) or 500 ng (tissue) was reverse transcribed using the high-capacity cDNA reverse transcriptase kit (ThermoFisher Scientific, USA). Quantitative PCR was performed on a QuantStudio 6 machine (Applied Biosystems, United States) using Taqman mastermix (Applied Biosystems, United States) and customized TaqMan FAM-TAMRA primers and probes purchased from IDT (Table 4). Gene copy numbers were normalized to housekeeping gene 18S and quantitated using a standard curve.

TABLE 4
Nucleotide sequences of TaqMan primers and probes

| Gene | Forward | Reverse | Probe |
|---|---|---|---|
| 18S | CGCCGCTAGAGGTGA | CATTCTTGGCAAAT | FAMACCGGCGCAAG |
| | AATTCT | GCTTTCG | ACGGACCAGA- |
| | | | TAMRA |
| IL-25 | GAGATATGAGTTGGA | CCATGTGGGAGCCT | FAMCTCCCCCAGGA |
| | CAGAGACTTGAA | GTCTGTA | CCTGTACCACGC- |
| | | | TAMRA |
| IL-17RB | GCTCTTATCCAACACA | CACCGTAGCACCTT | FAMAGCCACACCAG |
| | GCACTA | CACTATC | AAGAAACAAACGC- |
| | | | TAMRA |
| RV-A1 | GTGAAGAGCCsCrTGT | GCTsCAGGGTTAAG | FAMTGAGTCCTCCG |
| | GCT | GTTAGCC | GCCCCTGAATG- |
| | | | TAMRA |
| 229E | CAGTCAAATGGGCTG | AAAGGGCTATAAA | FAMCCCTGACGACC |
| | ATGCA | GAGAATAAGGTATT | ACGTTGTGGTTCA- |
| | | CT | TAMRA |
| IFN-ß | CGCCGCATTGACCATC | TTAGCCAGGAGGTT | FAMTCAGACAAGAT |
| | TA | CTCAACAATAGTCT | TCATCTAGCACTGG |
| | | CA | CTGGA-TAMRA |
| IL-λ2/3 | CTGCCACATAGCCCA | AGAAGCGACTCTTC | FAMTCTCCACAGGA |
| | GTTCA | TAAGGCATCTT | GCTGCAGGCCTTTA- |
| | | | TAMRA |

All sequences presented in 5′ to 3′ orientation.

### (6) Immune Transcriptome Analysis

Extracted RNA was hybridized to the human immunology v2 GX code set (Nanostring, United States) as per manufacturer's instructions with 50 ng of RNA. Raw data was imported into the Nanostring nCounter advanced analysis software v2.0 for normalisation based on positive and negative controls, and GENorm selection of housekeeper genes. Normalised data were then exported and graphed as digital mRNA counts for exact copy number data or was expressed as log 2 fold change ratio against-log 10 Bejamini-Yekutieli-corrected p-values for volcano plot data visualisation.

### (7) Cytokine and Chemokine Protein Analysis

BEC ALI supernatants were assessed for IFN-21/3, (R&D, United States) and IFN-β (PBL assay sci, United States) protein expression by ELISA, as per the manufacturer's instructions. Human IL-25 was measured in BEC apical media and cell lysates by ELISA (Abeome, United States). Cell debris from protein lysates was removed by centrifugation at 9000×g for 10 minutes at 4° C. and protein concentration was determined by BCA assay (ThermoFisher, USA).

96 well plates were coated with LNR 126 capture antibody in PBS and stored at 4° C. overnight. Plates were washed 3X in PBS and 0.075% tween-20 and blocked with reagent diluent (R&D systems, USA) at room temperature for one hour. The standard was prepared with recombinant IL-25 (R&D Systems) with a range of 250-1.95 pg/mL in reagent diluent. Samples were diluted in a 1:4 combination of RIPA buffer and reagent diluent. Biotinylated LNR125 detection antibody in blocking buffer and incubated 1 hour at room temperature. For detection 1-step Ultra TMB reagent (thermofisher, USA) was added. Readings were taken at 5, 10, 15, 20, and 30 minutes at 633 nm during development with gentle agitation before each reading. Peak absorbance readings were taken and normalized to BCA protein concentration.

(8) Mouse IL-4, IL-5, IL-13, IL-25, IFN-β, and IFN-λ2/3 were Quantified by Duoset ELISA (R&D Systems, United States).

Cytokine and chemokine quantification of conditionally reprogrammed cells for IFN-β, IFN-λ2/3, IFN-λ1, IFN-γ, IFN-α2, CXCL10, IL-1β, and IL-6 was measured using the LEGENDplex human anti-virus response multiplex flow cytometry panel (Biolegend, United States) as per the manufacturer's instructions. Data was acquired with the FACS Canto II (Beckman Coulter; United States) and analysed with LEGENDplex v8.0 software (Biolegend, United States).

### (9) Immunofluorescence

Paraffin embedded endoscopic lung biopsies and ALI sections were deparaffinised in xylene, then rehydrated in ethanol before being subjected to antigen retrieval in sodium citrate buffer. Slides were washed in TBS-T then were blocked for 1 hour in 5% donkey serum/5% casein solution in TBS-T in a humidified chamber. The blocked sections were incubated overnight at 4° C. with anti-IL-17RB (MAB1207, R&D Systems) and anti-IL-25 (BAF1258, R&D Systems, USA) in2% donkey serum/2% casein in TBS-T. Primary antibodies were washed off and HRP-488 (405235, Biolegend, USA) and Alexa-fluor-594 (SAB4600407, Sigma Aldrich, USA) were applied in the dark for 2 hours at room temperature. Secondary antibodies were washed off and mountant containing DAPI was applied prior to imaging with the Axio imager M2 fluorescent microscope and analysing with Zen 2 software (Carl Zeiss AG, Germany).

(10) Western blot

Cell debris from protein lysates was removed by centrifugation at 9000×g for 10 minutes at 4° C. and protein concentration was determined by BCA assay (ThermoFisher, USA). Five micrograms of protein was resolved by 4-15% TGX Stain free SDS PAGE gel (Biorad, USA) and semi-dry transferred onto nitrocellulose (Biorad, USA). Membranes were blocked in 3% BSA 2% skim milk in TBS-T for 1 hour at room temperature, then incubated overnight at 4° C. with anti-IL-17RB or β-actin (ab8227, Abcam). After extensive washing, membranes were incubated with HRP-conjugated secondary antibodies for 1 hour. Membranes were developed with Supersignal west femto (ThermoFisher, USA) and imaged on a Chemi-Doc (Biorad, USA).

### (11) Statistical Analyses

Results and error bars were presented as +/−standard deviation (s.d.) or median +/−interquartile range (IQR). In all experiments, n represents the number of individual pBECs donors or mice. Wilcoxon matched-pairs T-tests was applied to determine statistical significance for differences between two groups. Parametric in vitro data were analysed using Friedmen multiple comparisons test or one-way ANOVA where applicable Holm-Sidak's correction for multiple analysis. Parametric mouse data were analysed using one-way or two-way ANOVA where appropriate with Holm-Sidak's correction for multiple analyses. Non-parametric mouse data was analysed by a Mann-Whitney test. Statistical significance was set at *p<0.05, ** p<0.01, *** p<0.001, **** p<0.0001. Analyses were performed using GraphPad Prism v8.3 software (GraphPad, United States).

### c) Discussion

Epithelial cell-expressed IL-25 promotes allergic diseases such as asthma and is increased during viral asthma exacerbations. IL-25 stimulates type 2 inflammation and contributes to airway obstruction by triggering bronchoconstriction, mucus production and infiltration of inflammatory cells into airways. The need for efficient control of infection by airway epithelial cells to limit the capacity of viruses to provoke airway inflammation in the context of asthma exacerbations has led to the discovery epithelial cell-intrinsic delayed and deficient anti-viral immunity. IL-25 is constitutively expressed by airway epithelial cells making it a candidate for regulating epithelial cell innate immunity which led us to investigate the role of IL-25 on antiviral immunity in asthma. First, we confirmed that IL-25 is constitutively expressed in vivo in human airways (endobronochial biopsies) and this was replicated in differentiated primary human BECs. We noted abundant constitutive expression of IL-25 protein at the apical surface airway epithelial cells from both healthy donors and subjects with asthma. A similar expression pattern was noted for the IL-25 receptor with evidence of colocalization of IL-25 and IL-17RB supporting a role for IL-25 autocrine/paracrine signaling in directly regulating epithelial cell mediated mucosal immunity, in addition to indirectly via activation of mucosal-resident innate immune cells such as type 2 innate lymphoid cells and plasmacytoid dendritic cells. Despite the abundant constitutive expression, we were able to detect increased IL-25 mRNA and protein (cell associated) expression by RV infection of ALI-BECs derived from asthmatic donors. We could not reliably detect secreted IL-25 in apical media consistent with substantial binding of IL-25 to the apical surface of epithelial cells. A recent study in nasal epithelial cells has reported that IL-25 is induced during Influenza A infection, and that pre-treatment with IFN-α reduced IL-25 gene expression suggesting that type I IFN antagonised virus-induced epithelial cell IL-25.

To determine if the reciprocal is true-IL-25 negatively regulates anti-viral responses-we used a potent anti-IL-25 mAb (LNR125) to determine if blocking IL-25 produced by BECs from patients with moderate to severe asthma augmented IFN production during RV infection. To gain an overview of the effect on BEC innate immunity we used Nanostring Immune transcriptomic analyses and indicated that blocking IL-25 promoted expression of innate anti-viral interferons (type I/III IFNs) and ISG expression whilst suppressing expression of immune pathways that antagonise anti-viral immunity-type 2 immunity and TGF-β. Multiple mechanisms by which type 2 immune pathways interfere with innate anti-viral responses in the context of asthma have been described. This is the first study to provide evidence that a therapeutic anti-IL-25 mAb directly boosts airway epithelial cell anti-viral immunity and points to IL-25 blockade as being particularly beneficial for viral asthma exacerbations where a therapy with dual activity-block type 2 inflammation and promote anti-viral immunity-would be desirable.

Biologics (mAbs) specifically targeting type 2 immune pathways have provided clinical benefit in reducing frequency of asthma exacerbations and there are now several approved for use in asthma including omalizumab (anti-IgE mAb), dupilumab (anti-IL-4Rα), mepolizumab and reslizumab (anti-IL-5) and benralizumab (anti-IL-5R). The use of these biologics is limited to asthmatics who cannot control symptoms with corticosteroids and satisfy immunological criteria: omalizumab is approved for use in atopic asthma (high IgE). For the type 2 cytokine-targeting mAbs there must be elevated blood eosinophils. Based on these criteria many patients with severe, difficult to treat asthma are not eligible for treatment with these mAbs highlighting the need for further research into understanding the immunological aetiology of different disease phenotypes/endotypes in severe asthma. Since the majority of severe asthma exacerbations are triggered by a respiratory virus infection³, targeting cytokines that emanate from infected airway epithelium has become a focus. Three such cytokines-IL-25, IL-33 and TSLP have been of particular interest given their role in stimulating airway inflammation in models of severe asthma and asthma exacerbations with increasingly detailed dissection of their functions revealing distinct effects on specific immune cell populations. So far only anti-TLSP (tezepelumab) has reported phase 2 clinical data showing reduction in frequency of asthma exacerbations.

Less progress has been made with treatments that stimulate anti-viral immunity. Inhaled IFN-β has previously been tested in a clinical trial for individuals with asthma during respiratory viral infections. Assessment of the clinical benefit of inhaled IFN-β was confounded by the lack of virus induced exacerbations detected in this study and therefore a reduction of asthma symptoms was not detected. Analysis of a subgroup with severe disease did show IFN-β treatment reduced symptom severity. One potential reason for the limited efficacy of inhaled IFN-β is limited duration of in vivo bioactivity of the recombinant protein. Blocking IL-25 is evidence to support innate immune-targeting approaches that ‘re-calibrate’ mucosal innate immunity to improve the capacity for endogenous IFN production during viral infection. Other innate immune-stimulating approaches support this strategy. We have reported that TLR2-agonist innate immune priming also re-calibrates BEC response to RV infection, characterised by rapid NF-κB activation and IFN-λ production that enhances control of viral infection in vitro and in vivo.

We showed that LNR 125-IL-25 blockade increased IFN-β and IFN-λ mRNA and protein express by RV infected BECs from subjects with moderate to severe asthma, a disease phenotype associated with deficient virus-induced epithelial cell IFN production. The focus of this study was to understand the effect of IL-25 on anti-viral immunity in asthma to determine therapeutic potential of IL-25 blockade for viral asthma exacerbations. Therefore, we did not compare IL-25 blockade and anti-viral responses of BECs from asthmatic subjects with BECs from healthy donors. We did note that that the RV-infected asthmatic BECs used in this study, treated with isotype control antibody, failed to significantly increase expression of type I/III IFNs by 4 day post-infection which is consistent with a deficient response, that can be improved by mAb-mediated inhibition of IL-25. Immune transcriptome analysis of LNR 125 treated BECs from individuals with asthma indicated that blocking IL-25 restored antiviral immunity with evidence of upregulation of ISGs such as IFN regulatory factor 7 (IRF7), TANK binding kinase 1 (TBK1), and interleukin-1 receptor associated kinase 2 (IRAK2). Protein validation of total and phosphorylated TBK1 and IRF7 by immunoblot could not detect a difference in protein expression and therefore did not provide confirmatory evidence for the transcriptomic data. We attributed this to the low sensitivity of western blot for quantifying transcription factors, particularly in ALI-differentiated BEC-RV infection models in which only a small number of cells are infected. Indeed, “patchy” viral infection has been reported in airway epithelial cells during respiratory viral infection. BECs infected with the coronavirus strain 229E, IL-25 blockade enhanced IFN-λ expression. We examined LNR125 treatment in healthy BECS infected with an endemic coronavirus strain 229E. We observed enhanced production of IFN-22/3. Further, we found a trend in up-regulation of IFN-λ1, IFN-β, IFN-α2, and IFN-γ. We can conclude that treatment with LNR125 induced an earlier innate immune response in healthy BECs infected with 229E, implicating a potential role for IL-25 blockade in boosting anti-viral immunity during coronavirus infection.

In addition to IFNs, we found a trend towards increased IL-1β, a component of the inflammasome which can suppress IL-25 cytokine production during helminth infection. Inversely, IL-25 has been reported to be a negative regulator of proinflammatory cytokines secretion of IL-1β, TNF-α, and IL-6 In monocytes. This data implicates IL-25 suppression of antiviral immunity extends to multiple respiratory viruses including coronavirus, RV and influenza. We also examined the induction of IL-25 and effects on antiviral immunity during CoV 229E infection. Unlike RV, we did not observe 229E-increased IL-25 expression by differentiated primary human BECs, although blocking IL-25 did increase virus-induced IFN-λ. This observation is consistent with RV having greater capacity to promote type 2 inflammation and might underlie the greater prevalence of this virus in triggering asthma exacerbations.

IL-25 or IL-17RB blockade during respiratory viral infection in vivo has reduced immune cell infiltration and pro-inflammatory mediators in the BAL. Using a mouse model of RV-exacerbation of allergic airway disease, we found LNR125 reduced total BAL cell counts, and secretion of IL-4, IL-5, and IL-25. In contrast to ALI-BEC, we found LNR 125 upregulated IFN-β, but not IFN-λ. IFN-β has been shown to upregulate IFN-λ expression. Therefore, the absence of an effect on IFN-λ can be attributed to the early time point of analysis (one day post-infection) in this in vivo model. IFN-γ is suppressed by IL-25 during helminth infection, however we are the first to show IL-25 regulates epithelial derived IFNs during allergic inflammation.

IL-25 induces and amplifies type-2 inflammation through activation and recruitment of lymphocytes and granulocytes. Moreover, a feed-forward mechanism has been identified within human lung tissue where IL-4 upregulated IL-25 and IL-17RB expression. We found LNR125 treatment reduced IL-17RB expression within our in vitro model, indicating that LNR125 treatment could disrupt this feed-forward propagation of type-2 inflammation. In our mouse model, we observed LNR125 reduced total BAL, lymphocytes, and eosinophil recruitment 7 days p.i., supporting the role of IL-25 in amplifying type-2 inflammation. During an RV exacerbation of allergic airways disease, LNR 125 treatment inhibited expression of IL-4, IL-5, and IL-25, which is entirely consistent with a previous study in this model using mAb blockade of the IL-25 receptor (IL-17RB).

Increased inflammation in asthma has been associated with airway remodelling and reduced BEC differentiation. One insight from the IL-25 blockade transcriptomic data in BECs from donors with asthma highlighted that IL-25 regulates TGF-β signaling; a contributor to airway remodelling. While IL-25 blockade did not regulate any isoforms of TGF-β, we found LNR125 treatment reduced the inducer of TGF-β (TGF-βI), while upregulating the negative regulator SKI. This implicates that IL-25 can indirectly regulate TGF-β expression. A study conducted in pBECs treated with an anti-TGF-β antibody reported increased IFN-β and IFN-λ1 production when stimulated with poly IC (a viral mimic). Therefore, the enhanced IFN secretion we report with LNR125 can be through multiple mechanisms such as suppression of TGF-β signaling, in addition to the enhanced ISG expression.

In conclusion we show that IL-25 blockade improved antiviral immunity during respiratory viral infection in individuals with asthma. In BECs from donors with asthma, this was through inhibiting the suppressive effects of IL-25 on IFN production and ISG expression and reducing type-2 inflammation. Our allergic in vivo model further showed reductions in type 2 cytokines and IL-25 in the lung associated with increased IFN-β production and reduced lung viral load. Finally, we showed that IL-25 blockade during coronavirus infection upregulated IFN-λ2/3. Therefore, IL-25-induced airway inflammation combined with suppression of epithelial cell anti-viral immunity identify IL-25 as central mediator of viral asthma exacerbations.

### D. References

Albers F C, et al. Biologic treatment eligibility for real-world patients with severe asthma: The IDEAL study. J Asthma 55, 152-160 (2018).
Ank N, West H, Bartholdy C, Eriksson K, Thomsen A R, Paludan S R. Lambda interferon (IFN-lambda), a type III IFN, is induced by viruses and IFNs and displays potent antiviral activity against select virus infections in vivo. J Virol 80, 4501-4509 (2006).
Barlow J L, et al. IL-33 is more potent than IL-25 in provoking IL-13-producing nuocytes (type 2 innate lymphoid cells) and airway contraction. J Allergy Clin Immunol, (2013).
Barlow J L, et al. Innate IL-13-producing nuocytes arise during allergic lung inflammation and contribute to airways hyperreactivity. J Allergy Clin Immunol 129, 191-198 e191-194 (2012).
Bartlett N W, et al. Mouse models of rhinovirus-induced disease and exacerbation of allergic airway inflammation. Nat Med 14, 199-204 (2008).
Beale J, et al. Rhinovirus-induced IL-25 in asthma exacerbation drives type 2 immunity and allergic pulmonary inflammation. Sci Transl Med 6, 256ra134 (2014).
Bedke N, et al. Transforming growth factor-beta promotes rhinovirus replication in bronchial epithelial cells by suppressing the innate immune response. PLOS ONE [Electronic Resource] 7, e44580 (2012).
Bel E H, et al. Oral glucocorticoid-sparing effect of mepolizumab in eosinophilic asthma. N Engl J Med 371, 1189-1197 (2014).
Boxall C, Holgate S T, Davies D E. The contribution of transforming growth factor-beta and epidermal growth factor signaling to airway remodelling in chronic asthma. Eur Respir J 27, 208-229 (2006).
Busse W W, et al. Randomized trial of omalizumab (anti-IgE) for asthma in inner-city children. N Engl J Med 364, 1005-1015 (2011).
Caruso R, et al. Inhibition of monocyte-derived inflammatory cytokines by IL-25 occurs via p38 Map kinase-dependent induction of Socs-3. Blood 113, 3512-3519 (2009).
Castro M, et al. Benralizumab, an anti-interleukin 5 receptor alpha monoclonal antibody, versus placebo for uncontrolled eosinophilic asthma: a phase 2b randomised dose-ranging study. The Lancet Respiratory medicine 2, 879-890 (2014).
Castro M, et al. Dupilumab Efficacy and Safety in Moderate-to-Severe Uncontrolled Asthma. N Engl J Med 378, 2486-2496 (2018).
Castro M, et al. Reslizumab for inadequately controlled asthma with elevated blood eosinophil counts: results from two multicentre, parallel, double-blind, randomised, placebo-controlled, phase 3 trials. Lancet Respir Med 3, 355-366 (2015).
Castro M, et al. Reslizumab for poorly controlled, eosinophilic asthma: a randomized, placebo-controlled study. Am J Respir Crit Care Med 184, 1125-1132 (2011).
Castro M, Zangrilli J, Wechsler M E. Corrections. Reslizumab for inadequately controlled asthma with elevated blood eosinophil counts: results from two multicentre, parallel, double-blind, randomised, placebo-controlled, phase 3 trials. Lancet Respir Med 3, e15 (2015).
Chairakaki A D, et al. Plasmacytoid dendritic cells drive acute asthma exacerbations. J Allergy Clin Immunol, (2017).
Contoli M, et al. Role of deficient type III interferon-lambda production in asthma exacerbations. Nat Med 12, 1023-1026 (2006).
Corren J, et al. Tezepelumab in Adults with Uncontrolled Asthma. N Engl J Med 377, 936-946 (2017).
Corrigan C J, et al. Allergen-induced expression of IL-25 and IL-25 receptor in atopic asthmatic airways and late-phase cutaneous responses. J Allergy Clin Immunol 128, 116-124 (2011).
Corrigan C J, et al. T-helper cell type 2 (Th2) memory T cell-potentiating cytokine IL-25 has the potential to promote angiogenesis in asthma. Proc Natl Acad Sci USA 108, 1579-1584 (2011).
Denney L, Branchett W, Gregory L G, Oliver R A, Lloyd C M. Epithelial-derived TGF-beta1 acts as a pro-viral factor in the lung during influenza A infection. Mucosal Immunol 11, 523-535 (2018).
Djukanovic R, et al. The effect of inhaled IFN-beta on worsening of asthma symptoms caused by viral infections. A randomized trial. Am J Respir Crit Care Med 190, 145-154 (2014).
Durrani S R, et al. Innate immune responses to rhinovirus are reduced by the high-affinity IgE receptor in allergic asthmatic children. J Allergy Clin Immunol 130, 489-495 (2012).
Fahy J V. Type 2 inflammation in asthma—present in most, absent in many. Nat Rev Immunol 15, 57-65 (2015).
Fallon P G, et al. Identification of an interleukin (I L)-25-dependent cell population that provides IL-4, IL-5, and IL-13 at the onset of helminth expulsion. J Exp Med 203, 1105-1116 (2006).
Fort M M, et al. IL-25 induces IL-4, IL-5, and IL-13 and Th2-associated pathologies in vivo. Immunity 15, 985-995 (2001).
Gentzsch M, et al. Pharmacological Rescue of Conditionally Reprogrammed Cystic Fibrosis Bronchial Epithelial Cells. Am J Respir Cell Mol Biol 56, 568-574 (2017).
Girkin J, et al. TLR2-mediated innate immune priming boosts lung anti-viral immunity. Eur Respir J, (2020).
Hackett T L, et al. Characterization of side population cells from human airway epithelium. Stem Cells 26, 2576-2585 (2008).
Hackett T L, et al. Induction of epithelial-mesenchymal transition in primary airway epithelial cells from patients with asthma by transforming growth factor-beta1. Am J Respir Crit Care Med 180, 122-133 (2009).
Haldar P, et al. Mepolizumab and exacerbations of refractory eosinophilic asthma. N Engl J Med 360, 973-984 (2009).
Hanania N A, et al. Lebrikizumab in moderate-to-severe asthma: pooled data from two randomised placebo-controlled studies. Thorax 70, 748-756 (2015).
Hatchwell L, et al. Toll-like receptor 7 governs interferon and inflammatory responses to rhinovirus and is suppressed by IL-5-induced lung eosinophilia. Thorax 70, 854-861 (2015).
Holt P G, Sly P D. Viral infections and atopy in asthma pathogenesis: new rationales for asthma prevention and treatment. Nat Med 18, 726-735 (2012).
Honda K, Yanai H, Takaoka A, Taniguchi T. Regulation of the type I IFN induction: a current view. Int Immunol 17, 1367-1378 (2005).
Hong H, et al. Induction of IL-25 Expression in Human Nasal Polyp Epithelium by Influenza Virus Infection is Abated by Interferon-Alpha Pretreatment. J Inflamm Res 14, 2769-2780 (2021).
Hurst S D, et al. New IL-17 family members promote Th1 or Th2 responses in the lung: in vivo function of the novel cytokine IL-25. J Immunol 169, 443-453 (2002).
Johnston S L, et al. The relationship between upper respiratory infections and hospital admissions for asthma: a time-trend analysis. Am J Respir Crit Care Med 154, 654-660 (1996).
Kaiko G E, Phipps S, Angkasekwinai P, Dong C, Foster P S. N K cell deficiency predisposes to viral-induced Th2-type allergic inflammation via epithelial-derived IL-25. J Immunol 185, 4681-4690 (2010).
Kouzaki H, Tojima I, Kita H, Shimizu T. Transcription of interleukin-25 and extracellular release of the protein is regulated by allergen proteases in airway epithelial cells. Am J Respir Cell Mol Biol 49, 741-750 (2013).
Liu X, et al. ROCK inhibitor and feeder cells induce the conditional reprogramming of epithelial cells. Am J Pathol 180, 599-607 (2012).
Loo S L, Wark PAB, Esneau C, Nichol K S, Hsu A C, Bartlett N W. Human coronaviruses 229E and OC43 replicate and induce distinct antiviral responses in differentiated primary human bronchial epithelial cells. Am J Physiol Lung Cell Mol Physiol 319, L926-L931 (2020).
Martinovich K M, et al. Conditionally reprogrammed primary airway epithelial cells maintain morphology, lineage and disease specific functional characteristics. Sci Rep 7, 17971 (2017).
Mosser A G, et al. Similar frequency of rhinovirus-infectible cells in upper and lower airway epithelium. J Infect Dis 185, 734-743 (2002).
Nair P, Barker P, Goldman M. Glucocorticoid Sparing of Benralizumab in Asthma. N Engl J Med 377, 1205 (2017).
Nair P, et al. Oral Glucocorticoid-Sparing Effect of Benralizumab in Severe Asthma. N Engl J Med 376, 2448-2458 (2017).
Nicholson K G, Kent J, Ireland D C. Respiratory viruses and exacerbations of asthma in adults. Bmj 307, 982-986 (1993).
Ortega H G, et al. Mepolizumab treatment in patients with severe eosinophilic asthma. N Engl J Med 371, 1198-1207 (2014).
Ortega H G, et al. Mepolizumab treatment in patients with severe eosinophilic asthma. N Engl J Med 371, 1198-1207 (2014).
Owyang A M, et al. Interleukin 25 regulates type 2 cytokine-dependent immunity and limits chronic inflammation in the gastrointestinal tract. J Exp Med 203, 843-849 (2006).
Pavord I D, et al. Mepolizumab for severe eosinophilic asthma (DREAM): a multicentre, double-blind, placebo-controlled trial. Lancet 380, 651-659 (2012).
Petersen B C, Dolgachev V, Rasky A, Lukacs N W. IL-17E (IL-25) and IL-17R B promote respiratory syncytial virus-induced pulmonary disease. J Leukoc Biol 95, 809-815 (2014).
Piper E, et al. A phase I I placebo-controlled study of tralokinumab in moderate-to-severe asthma. Eur Respir J 41, 330-338 (2013).
Saenz S A, et al. IL-25 simultaneously elicits distinct populations of innate lymphoid cells and multipotent progenitor type 2 (MPPtype2) cells. J Exp Med 210, 1823-1837 (2013).
Tamachi T, et al. IL-25 enhances allergic airway inflammation by amplifying a TH2 cell-dependent pathway in mice. J Allergy Clin Immunol 118, 606-614 (2006).
Tay H, Wark P A, Bartlett N W. Advances in the treatment of virus-induced asthma. Expert Rev Respir Med 10, 629-641 (2016).
Teach S J, et al. Preseasonal treatment with either omalizumab or an inhaled corticosteroid boost to prevent fall asthma exacerbations. J Allergy Clin Immunol 136, 1476-1485 (2015).
Vareille M, Kieninger E, Edwards M R, Regamey N. The airway epithelium: soldier in the fight against respiratory viruses. Clin Microbiol Rev 24, 210-229 (2011).
Veerati P C, et al. Airway Epithelial Cell Immunity Is Delayed During Rhinovirus Infection in Asthma and COPD. Front Immunol 11, 974 (2020).
Wang H, et al. Allergen challenge of peripheral blood mononuclear cells from patients with seasonal allergic rhinitis increases IL-17R B, which regulates basophil apoptosis and degranulation. Clin Exp Allergy 40, 1194-1202 (2010).
Wark P A, et al. Asthmatic bronchial epithelial cells have a deficient innate immune response to infection with rhinovirus. J Exp Med 201, 937-947 (2005).
Weathington N M, et al. IL-4 Induces the IL17Rb Gene Transcription in Monocytic Cells with Coordinate Autocrine IL-25 Signaling. Am J Respir Cell Mol Biol, (2017).
Wenzel S, et al. Dupilumab in persistent asthma with elevated eosinophil levels. N Engl J Med 368, 2455-2466 (2013).
Wynn T A. Type 2 cytokines: mechanisms and therapeutic strategies. Nat Rev Immunol 15, 271-282 (2015).
Zaiss M M, Maslowski K M, Mosconi I, Guenat N, Marsland B J, Harris N L. IL-1beta suppresses innate IL-25 and IL-33 production and maintains helminth chronicity. PLOS Pathog 9, e1003531 (2013).
Zhang Y, Wang Y, Li M Q, Duan J, Fan D X, Jin L P. IL-25 promotes Th2 bias by upregulating IL-4 and IL-10 expression of decidual gammadeltaT cells in early pregnancy. Exp Ther Med 15, 1855-1862 (2018).
Zhou B, et al. Thymic stromal lymphopoietin as a key initiator of allergic airway inflammation in mice. Nat Immunol 6, 1047-1053 (2005).

E. Sequences

SEQ ID NO: 1 anti-IL25 heavy chain CDR1 amino
acid sequence
STSGMGVG
SEQ ID NO: 2 anti-IL25 heavy chain CDR2 amino
acid sequence
HIWWDDVKRYNPALKS
SEQ ID NO: 3 anti-IL25 heavy chain CDR3 amino
acid sequence
TLPHFHDY
SEQ ID NO: 4 anti-IL25 heavy chain variable
domain
QVTLKVSGPGILQPSQTLSLTCSFSGFSLNTSGMGVGWIRQPSGKGLEW
LAHIWWDDVKRYNPALKSRLTISKDTSGSQVFLKIASVDTADTATYYCA
RTLPHFFDYWGQGTTLTVSS
SEQ ID NO: 5 anti-IL25 light chain CDR1 amino
acid sequence
RASSSVSYMY
SEQ ID NO: 6 anti-IL25 light chain CDR2 amino
acid sequence
RTSNLAS
SEQ ID NO: 7 anti-IL25 light chain CDR3 amino
acid sequence
KQYHSYPPTWT
SEQ ID NO: 8 anti-IL25 light chain variable
domain
DIQMTQSPAIMSASPGEKVTISCRASSSVSYMYWYQQKSGSSPKPWIYR
TSNLASGVPARFSGSGSGTSYSLTISSMEAEDAATYYCKQYHSYPPTWT
FGGGTKLEIKR
SEQ ID NO: 9
GTGAAGAGCCsCrTGTGCT
SEQ ID NO: 10
GCTSCAGGGTTAAGGTTAGCC
SEQ ID NO: 11
TGAGTCCTCCGGCCCCTGAATG
SEQ ID NO: 12 anti-IL25 LNR125.38 heavy chain
variable domain
QVTLKESGPTLVKPTQTLTLTCTFSGFSLSTSGMGVGWIRQPPGKALEW
LAHIWWDDVKRYNPSLKSRLTITKDTSGSQVVLTMTNMDPVDTATYYCA
RTLPHFHDYWGQGTTLTVSS
SEQ ID NO: 13 anti-IL25 LNR125.38 light chain
variable domain
DIQMTQSPSTLSASVGDRVTITCRASSSVSYMYWYQQKPGKAPKPLIYR
TSNLASGVPSRFSGSGSGTEYTLTISSLQPDDFATYYCKQYHSYPPTWT
FGGGTKVEIKR
SEQ ID NO: 32 anti-IL25 LNR125.1 grafted heavy
chain variable domain
QVTLKESGPTLVKPTQTLTLTCTFSGFSLNTSGMGVGWIRQPPGKALEW
LAHIWWDDVKRYNPSLKSRLTITKDTSGSQVVLTMTNMDPVDTATYYCA
RTLPHFFDYWGQGTTLTVSS
SEQ ID NO: 33 anti-IL25 LNR125.1 grafted light
chain variable domain
DIQMTQSPSTLSASVGDRVTITCRASSSVSYMYWYQQKPGKAPKPLIYR
TSNLASGVPSRFSGSGSGTEYTLTISSLQPDDFATYYCKQYHSYPPTWT
FGGGTKLEIKR
SEQ ID NO: 34 Motavizumab heavy chain variable
domain
QVTLRVSGPALVKPTQTLTLTCTFSGFSLSTAGMSVGWIRQPPGKALEW
LADIWWDDKKHYNPSLKDRLTISKDTSKNQVVLKVTNMDPADTATYYCA
RDMIFNFYFDVWGQGTTVTVSS
SEQ ID NO: 35 Motavizumab light chain variable
domain
DIQMTQSPSTLSASVGDRVTITCSASSRVGYMHWYQQKPGKAPKLLIYD
TSKLASGVPSRFSGSGSGTEFTLTISSLQPDDFATYYCFQGSGYPFTFG
GGTKVEIKR
SEQ ID NO: 36 Palivizumab heavy chain variable
domain
QVTLRVSGPALVKPTQTLTLTCTFSGFSLSTSGMSVGWIRQPPGKALEW
LADIWWDDKKDYNPSLKSRLTISKDTSANQVVLKVTNMDPADTATYYCA
RSMITNWYFDVWGAGTTVTVSS

What is claimed is:

1. An isolated anti-IL25 binding molecule comprising a heavy chain variable domain comprising a CDR1, CDR2, and CDR2 as set forth in SEQ ID NO: 1, SEQ ID NO: 2, and SEQ ID NO: 3, respectively.

2. The isolated anti-IL25 binding molecule of claim 1, comprising a heavy chain variable domain comprising the sequence as set forth in SEQ ID NO: 4 or SEQ ID NO: 12.

3. The isolated anti-IL25 binding molecule of claim 1 or 2, further comprising a light chain variable domain comprising a CDR1, CDR2, and CDR2 as set forth in SEQ ID NO: 5, SEQ ID NO: 6, and SEQ ID NO: 7, respectively.

4. The isolated anti-IL25 binding molecule of any of claims 1-3, comprising a light chain variable domain comprising the sequence as set forth in SEQ ID NO: 8 or SEQ ID NO: 13.

5. An isolated anti-IL25 binding molecule comprising a light chain variable domain comprising a CDR1, CDR2, and CDR2 as set forth in SEQ ID NO: 5, SEQ ID NO: 6, and SEQ ID NO: 7, respectively.

6. The isolated anti-IL25 binding molecule of claim 5, comprising a light chain variable domain comprising the sequence as set forth in SEQ ID NO: 8 or SEQ ID NO: 13.

7. A method of treating a rhinoviral infection, coronavirus infection, airway inflammation, rheumatoid arthritis, asthma, osteoarthritis, bone erosion, intraperitoneal abscesses and adhesions, inflammatory bowel disorder, allograft rejection, psoriasis, cancer, angiogenesis, atherosclerosis, cystic fibrosis and multiple sclerosis in a subject comprising administering to the subject a therapeutically effective amount of any of the IL-25 binding molecules of any of claims 1-6.
